(19) United States
(12) Patent Application Publication
(10) Pub. No.: US 2022/0011297 A1
(43) Pub. Date: Jan. 13, 2022

(54) METHOD FOR PREDICTING EFFECT OF IMMUNE CHECKPOINT INHIBITOR

(71) Applicants: NAGASAKI UNIVERSITY, Nagasaki-shi (JP); Foundation for Biomedical Research and Innovation at Kobe, Kobe-shi (JP)

(72) Inventors: Yoshimasa TANAKA, Tokyo (JP); Hiroaki SENJU, Tokyo (JP); Hiroshi MUKAE, Tokyo (JP); Masanori FUKUSHIMA, Tokyo (JP)

(73) Assignees: NAGASAKI UNIVERSITY, Nagasaki-shi (JP); Foundation for Biomedical Research and Innovation at Kobe, Kobe-shi (JP)

(21) Appl. No.: 17/282,637

(22) PCT Filed: Oct. 1, 2019
(86) PCT No.: PCT/JP2019/038721

§ 371 Date: Apr. 2, 2021

(30) Foreign Application Priority Data
Oct. 3, 2018 (JP) 2018-187856

(51) Int. Cl. G01N 33/50; C12N 5/0783

(52) U.S. Cl. CPC G01N 33/5094; C12N 5/0638; C12N 2501/999; G01N 2800/52; C12N 2501/2318; G01N 2800/12; G01N 2800/50; C12N 2501/2302

(57) ABSTRACT

A method may predict risk of onset of severe interstitial pneumonia caused by an immune checkpoint inhibitor to achieve a safe and highly effective cancer immunotherapy. Any one or more selected from: (a) cell count or proportion of Vδ2⁺γδ T cells in peripheral blood mononuclear cells isolated from a subject; (b) cell count or proportion of Vδ2⁺γδ T cells after antigenic stimulation in peripheral blood mononuclear cells isolated from a subject; (c) cell count or proportion of Vδ2⁺γδ T cells in peripheral blood T cells isolated from a subject; and (d) cell count or proportion of Vδ2⁺γδ T cells after antigenic stimulation in peripheral blood T cells isolated from a subject are measured, and the risk of onset of severe interstitial pneumonia is predicted by using the cell count or proportion as an index.

### RELATED APPLICATION

The present specification includes contents described in a specification of Japanese Patent Application No. 2018-187856 (filed on Oct. 3, 2018) on which priority of the present application is based.

### TECHNICAL FIELD

The present invention relates to a method for assessing whether a cancer immunotherapy with an immune checkpoint inhibitor would be appropriate or not, and a kit for the method.

### BACKGROUND ART

“Cancer immunotherapy” using an immune checkpoint inhibitor is believed to be promising as a next-generation standard cancer therapy. An immune checkpoint is a molecule that recognizes and eliminates cancer cells, controlling the natural immune defense system. PD-1, which is expressed on immune effector cells, binds to PD-L1 or PD-L2 expressed in antigen-presenting cells to negatively control the immune defense system, functioning as an immune checkpoint.

Most cancer cells have a system to avoid the immune defense system by controlling signals from T cells, and it is known that there is a correlation between expression of PD-L1, which is a PD-1 ligand, and poor prognosis (Non Patent Literature 1). Although development of an anti-cancer agent by PD-1 immune checkpoint inhibition using an anti-PD-1 antibody or the like is ongoing, much remains unknown about the mechanism of action and the objective response rate of single use of it is only 5 to 30%. On the other hand, cases with the onset of an adverse event such as interstitial pneumonia are found, and thus management after administration is also important.

In addition, biomarkers to predict effects of immune checkpoint inhibitors have been searched for, and there are reports that blood concentrations of immunoglobulin, CD5L, and gelsolin are available as effect assessment markers for anti-PD-1 antibodies (Patent Literature 1), and that a specific miRNA is available for prediction of sensitivity to PD-1 inhibitors (Patent Literature 2). However, a method for assessing whether treatment with an immune checkpoint inhibitor would be appropriate or not has not been established yet.

Prediction/evaluation of whether treatment with an immune checkpoint inhibitor would be appropriate or not increases the objective response rate and enables achievement of precision medicine, which delivers appropriate medicine to appropriate patients, hence being desirable in terms of medical administration, not to mention safety for patients.

### CITATION LIST

### Patent Literature

Patent Literature 1: WO 2010/001617
Patent Literature 2: Japanese Patent Laid-Open No. 2016-64989

### Non Patent Literature

Non Patent Literature 1: Hamanishi J. et al., Proc Natl Acad Sci USA. 2007 Feb. 27; 104(9):3360-5. Epub 2007 Feb. 21.

### SUMMARY OF INVENTION

### Technical Problem

An object of the present invention is to achieve a more safe and highly effective cancer immunotherapy by predicting the risk of onset of severe interstitial pneumonia caused by an immune checkpoint inhibitor and diagnosing whether treatment with the immune checkpoint inhibitor would be appropriate or not to increase the objective response rate.

### Solution to Problem

Immune checkpoints act in two steps in the immune system. One is an action in a priming phase to negatively control the differentiation of Th0 cells into Th1 cells, Th2 cells, Th17 cells, or the like accompanied by first recognition of an antigen on an antigen-presenting cell. CTLA-4/CD80/CD86 are known as immune checkpoints in this priming phase, and they determine whether T cells recognize a specific antigen or not. The other is an action in an effector phase, in which immune effector cells disorder tumor cells or infected cells. PD-1/PD-L1/PD-L2 are known as immune checkpoints in this phase, and they determine whether T cells damage tumor cells or infected cells or not. Examples of known immune effector cells having cytotoxic activity include CD8-positive T cells (killer cells), γδ T cells, and NK cells. NK cells allow γδ T cells and CD8-positive T cells to grow, γδ T cells and NK cells damage target cells and then present an antigen-presenting molecule (MHC class I and II) and an antigen-peptide complex on their cell surfaces, and in response to this αβ T cells are sensitized to acquire antigen specificity and damage tumor cells or infected cells.

Based on the idea that the effect of a PD-1 immune checkpoint inhibitor is associated with the number and function of effector cells in a patient, the present inventors examined the relationship between the number, growth capacity, and antitumor cytotoxic activity of effector cells in the peripheral blood of a cancer patient who received treatment with an anti-PD-1 antibody (nivolumab) and adverse events and the objective response rate, and found that the risk of onset of an adverse event such as severe interstitial pneumonia from a PD-1 immune checkpoint inhibitor can be predicted by measuring the cell count or proportion of γδ T cells that serve as effector cells (Vδ2⁺γδ T cells) in peripheral blood mononuclear cells. γδ T cells that serve as effector cells are known to express, after being stimulated, an antigen-presenting cell-related molecule such as HLA-DR, HLA-DQ, CD80, and CD86 to perform antigen presentation to αβ T cells, and suggested to act in a secondary priming phase. From this, the present inventors found that the finding in the present invention is applicable to severe interstitial pneumonia and so on caused not only by a PD-1 immune checkpoint inhibitor but also by a CTLA-4 inhibitor, which acts in the secondary priming phase, thus completing the present invention.

Specifically, the present invention provides [1] to [13] in the following.

[1] A method for predicting a risk of onset of severe interstitial pneumonia caused by an immune checkpoint inhibitor, the method including:

measuring any one or more selected from:

(a) cell count or proportion of Vδ2⁺γδ T cells in peripheral blood mononuclear cells isolated from a subject;
(b) cell count or proportion of Vδ2⁺γδ T cells after antigenic stimulation in peripheral blood mononuclear cells isolated from a subject;
(c) cell count or proportion of Vδ2⁺γδ T cells in peripheral blood T cells isolated from a subject; and
(d) cell count or proportion of Vδ2⁺γδ T cells after antigenic stimulation in peripheral blood T cells isolated from a subject, and

assessing the risk of onset of severe interstitial pneumonia based on the cell count or proportion.

The method can distinguish interstitial pneumonia that is relatively acute and involves diffuse alveolar damage (DAD) (severe interstitial pneumonia) from the other types of interstitial pneumonia (e.g., those involving organizing pneumonia (OP)) and predict the onset.

[2] The method according to [1], wherein if the cell count or proportion is equal to or more than a cutoff value, the subject is predicted to have a high risk of onset of severe interstitial pneumonia.
[3] The method according to [1], wherein if the cell count or proportion after antigenic stimulation is high and cells after antigenic stimulation aggregate, the subject is predicted to have a high risk of onset of severe interstitial pneumonia.
[4] A method for assessing whether treatment with an immune checkpoint inhibitor would be appropriate or not, wherein prediction is performed on the risk of onset of severe interstitial pneumonia by the method according to any one of [1] to [3], and whether treatment with an immune checkpoint inhibitor would be appropriate or not is assessed based on the prediction.
[5] The method according to any one of [1] to [4], wherein the antigenic stimulation of γδ T cells is carried out by using any one or more antigens selected from IL-2, phosphomonoester compounds, pyrophosphomonoester compounds, triphosphomonoester compounds, tetraphosphomonoester compounds, triphosphodiester compounds, tetraphosphodiester compounds, nitrogen-containing bisphosphonate compounds, alkylamines, alkyl alcohols, alkenyl alcohols, isoprenyl alcohol, and human-derived tumor cells.
[6] The method according to [5], wherein in addition to the antigenic stimulation, γδ T cells are stimulated by using any one or more selected from IL-18, IL-2, IL-7, IL-12, IL-15, IL-21, IL-23, interferon-γ, and peripheral blood-conditioned medium.
[7] The method according to any one of [1] to [6], wherein the cell count or proportion is measured by using flow cytometry or image cytometry.
[8] A kit for assessing whether treatment with an immune checkpoint inhibitor would be appropriate or not, the kit including (i) an anti-CD3 antibody and (ii) an anti-Vδ2 antibody.
[9] The kit according to [8], further including one or more selected from:
(iii) a pyrophosphomonoester derivative or a nitrogen-containing bisphosphonate derivative, and

### (iv) IL-18.

[10] A method for assisting diagnosis of a risk of onset of severe interstitial pneumonia caused by an immune checkpoint inhibitor, the method including:
measuring any one or more selected from:
   (a) cell count or proportion of Vδ2⁺γδ T cells in peripheral blood mononuclear cells isolated from a subject;
   (b) cell count or proportion of Vδ2⁺γδ T cells after antigenic stimulation in peripheral blood mononuclear cells isolated from a subject;
   (c) cell count or proportion of Vδ2⁺γδ T cells in peripheral blood T cells isolated from a subject; and
   (d) cell count or proportion of Vδ2⁺γδ T cells after antigenic stimulation in peripheral blood T cells isolated from a subject, wherein
the risk of onset of severe interstitial pneumonia is determined based on the cell count or proportion.
   [11] A method for predicting a risk of onset of severe interstitial pneumonia caused by an immune checkpoint inhibitor and treating with the immune checkpoint inhibitor, the method including:
measuring any one or more selected from:
   (a) cell count or proportion of Vδ2⁺γδ T cells in peripheral blood mononuclear cells isolated from a subject;
   (b) cell count or proportion of Vδ2⁺γδ T cells after antigenic stimulation in peripheral blood mononuclear cells isolated from a subject;
   (c) cell count or proportion of Vδ2⁺γδ T cells in peripheral blood T cells isolated from a subject; and
   (d) cell count or proportion of Vδ2⁺γδ T cells after antigenic stimulation in peripheral blood T cells isolated from a subject;

performing prediction on the risk of onset of severe interstitial pneumonia based on the cell count or proportion; and

performing treatment with the immune checkpoint inhibitor according to the prediction (e.g., performing administration of the immune checkpoint inhibitor with exclusion of subjects having a high risk of onset, or applying a predetermined measure to patients having a high risk of onset and performing administration of the immune checkpoint inhibitor).

[12] The method according to any of [1] to [7], [10], and [11], wherein the subject is a lung cancer patient.
[13] A pharmaceutical composition containing an immune checkpoint inhibitor, wherein the pharmaceutical composition is used for treating or preventing tumor with suppression of onset of severe interstitial pneumonia, and used for a subject assessed with the method according to any of [1] to [7] to have a low risk of onset of severe interstitial pneumonia.

In [1] to [13] above, the immune checkpoint inhibitor is preferably a PD-1 immune checkpoint inhibitor.

### Advantageous Effects of Invention

The present invention achieves precision medicine, which provides appropriate medicine to appropriate patients, by predicting, before administration, a risk of onset of severe interstitial pneumonia caused by an immune checkpoint inhibitor and assessing whether the treatment would be appropriate or not. The method of the present invention is less invasive to patients because the method can be performed with only a little peripheral blood collected from a patient, and can diagnose in a quick and simple manner by using flow cytometry or the like.

### BRIEF DESCRIPTION OF DRAWINGS

FIG. 1 shows results of flow cytometry analysis on proportions of γδ T cells in peripheral blood mononuclear cells from healthy individuals.

FIG. 2 shows results of flow cytometry analysis on proportions of γδ T cells in peripheral blood mononuclear cells from healthy individuals when growth induction was performed with PTA and IL-2 (left: Day 0, right: Day 11). (A) A healthy individual with the proportion of Vδ2-type γδ T cells on Day 0 being 5.57%, (B) a healthy individual with the proportion of Vδ2-type γδ T cells on Day 0 being 10.35%.

FIG. 3 shows results of flow cytometry analysis on proportions of γδ T cells in lung cancer patients.

FIG. 4 shows flow cytometry analysis results on proportions of γδ T cells in peripheral blood mononuclear cells from lung cancer patients when growth induction was performed with PTA and IL-2 (left: Day 0, right: Day 11). (A) A lung cancer patient with the proportion of Vδ2-type γδ T cells on Day 0 being 4.14%, (B) a lung cancer patient with the proportion of Vδ2-type γδ T cells on Day 0 being 2.91%, (C) a lung cancer patient with the proportion of Vδ2-type γδ T cells on Day 0 being 0.89%, (D) a lung cancer patient with the proportion of Vδ2-type γδ T cells on Day 0 being 0.78%.

FIG. 5 shows the growth of γδ T cells when peripheral blood mononuclear cells from healthy individuals were stimulated with Zol/IL-2 or Zol/IL-2/IL-18 (in the figure, left: control (medium), center: Zol/IL-2, right: Zol/IL-2/IL-18).

FIG. 6 shows the growth of NK cells when a CD3-negative fraction of peripheral blood mononuclear cells from healthy individuals was stimulated with IL-2 or IL-2/IL-18 (in the figure, top: IL-2, bottom: IL-2/IL-18).

FIG. 7 shows results of confirmation of the relationship between the NK cell growth inducibility of IL-2/IL-18 and growth induction for γδ T cells through mixed culture (in the figure, left: single culture of 76 T cells, center: single culture of NK cells, right: mixed culture of NK cells and γδ T cells. NK cells (red), γδ T cells (green)).

FIG. 8 shows the mechanism of growth induction for γδ T cells after antigenic stimulation.

FIG. 9 shows the concept of cancer immunotherapy with a PD1 immune checkpoint inhibitor and prediction of the effect thereof.

### DESCRIPTION OF EMBODIMENTS

### 1. Definitions

### “Immune Checkpoint Inhibitor”

An immune checkpoint inhibitor refers to a substance that inhibits an immune checkpoint such as the CTLA-4/CD80/CD86 signal transduction system and the PD-1/PD-L1/PD-L2 signaling system and thereby exhibits antitumor effect and anti-infective effect through suppression of immunoediting by viruses and so on.

“PD-1 (Programmed Death-1)” is expressed on surfaces of effector T cells and negatively controls the immune defense system by interacting with PD-L1 expressed on surfaces of tumor cells, thus being what is called an immune checkpoint. PD-1 has two ITIM (Immunoreceptor tyrosine-based inhibition motif) structures in the intracellular domain, and is believed to transmit an immunosuppressive signal through binding of SHIP-2 to the C-terminal side of PD-1.

A “PD-1 immune checkpoint inhibitor” refers to a substance that inhibits an immune checkpoint system in which PD-1 involves. Thereby, the “PD-1 immune checkpoint inhibitor” suppresses the immunoediting mechanism of tumor cells to exhibit antitumor effect, and exhibits anti-infective effect by suppressing immunoediting by viruses or pathogenic microorganisms.

In general, T cells recognize antigen peptide/MHC class I or MHC class II complex presented on antigen-presenting cells in a T cell receptor (TCR)-dependent manner. However, complete immune response is not evoked only by a signal from this TCR/antigen peptide/MHC complex, and for priming of T cells the CD28/CD80/CD86 signal system is needed (positive costimulatory signal) in addition to a signal from the TCR/antigen peptide/MHC complex. When the CTLA-4/CD80/CD86 signal system operates, by contrast, the activation of T cells is negatively controlled (negative costimulatory signal). That is, costimulatory signals by CD28 and CTLA-4 specify the first stage of determining whether T cells react with a specific antigen. In the effector phase, on the other hand, the ICOS/ICOSL signal system serves as a positive costimulatory signal and the PD-1/PD-L1/PD-L2 signal system serves as a negative costimulatory signal. That is, the PD-1/PD-L1 system functions as a negative signal system in the phase to determine whether T cells kill target cells or not.

Three candidate PD-1 immune checkpoint inhibitors are contemplated: an anti-PD-1 antibody, an anti-PD-L1 antibody, and an anti-PD-L2 antibody. First, the anti-PD-1 antibody blocks both the interaction between PD-1 and PD-L1 and the interaction between PD-1 and PD-L2. On the other hand, the anti-PD-L1 antibody blocks only the interaction between PD-1 and PD-L1, and the anti-PD-L2 antibody blocks only the interaction between PD-1 and PD-L2.

PD-1 is known to be expressed in activated immune effector T cells. PD-L1 and PD-L2 are known to be expressed in tumor cells that cause poor prognosis, and PD-L2 is also expressed in dendritic cells. Therefore, the anti-PD-L1 antibody can be expected to specifically enhance the antitumor effect of T cells by inhibiting the interaction between PD-1 expressed on activated T cells and PD-L1 expressed on tumor cells to block immunosuppression of T cells. On the other hand, the anti-PD-L2 antibody inhibits the interaction between PD-1 expressed on activated T cells and PD-L2 expressed on tumor cells, and hence not only blocks immunosuppression of T cells but also inhibits the interaction between PD-1 expressed on T cells and PD-L2 expressed on dendritic cells, possibly affecting priming of T cells, too. That is, in addition to cancer-specific action, the anti-PD-L2 antibody and anti-PD-1 antibody may exhibit other different action. Thus, the anti-PD-L1 antibody is theoretically expected to be the most cancer-specific as a PD-1 immune checkpoint inhibitor and even cause less adverse effect; however, the actual action is needed to be analyzed in detail based on clinical situations.

Examples of PD-1 immune checkpoint inhibitors that act in the effector phase among immune checkpoint inhibitors currently commercially available or under development include the anti-PD-1 antibodies nivolumab (Opdivo), pembrolizumab (Keytruda), and pidilizumab (CT-011); and the anti-PD-L1 antibodies atezolizumab (MPDL3280A/RG-7446), Durvalumab (MEDI4736), avelumab (MSB0010718C), and MED10680/AMP-514. Examples of immune checkpoint inhibitors currently commercially available or under development that act in another phase include the anti-CTLA-4 antibodies ipilimumab (MDX-010) and tremelimumab (CP675, 206); the anti-killer cell immunoglobulin-like receptor (KRI) antibody lirilumab (IPH2102/BMS-986015); the anti-CD137 antibodies urelumab (BMS-663513) and PF-05082566; the anti-LAG3 antibody BMS-986016; and the anti-OX40 antibody MEDI6469.

“Interstitial Pneumonia”

Interstitial pneumonia is a collective term for diseases in which inflammatory and fibrotic lesions exist in the pulmonary interstitium, and a disease involving the fibrillization of the lung as a result of the progression of interstitial pneumonia is called pulmonary fibrosis. Interstitial pneumonia has a wide range of causes, and there are occupation/environment-specific or drug-induced ones, ones that develop in association with systemic diseases such as collagen disorder and sarcoidosis, and ones of unspecified causes. As a general tendency, the acute onset presents as diffuse alveolar damage (DAD) or the like as a clinical manifestation, and, on the other hand, the chronic onset presents as organizing pneumonia (OP) as a clinical manifestation. While OP and the like are generally benign and ameliorated in many cases by drug discontinuation or by the use of corticosteroid (steroid) however, DAD has poor treatment responsiveness, and results in poor prognosis and leaves fibrillization even after recovery.

“Severe Interstitial Pneumonia”

Herein, severe interstitial pneumonia means relatively acute interstitial pneumonia involving DAD, meaning symptoms of interstitial pneumonia that cause acute exacerbation and may lead to death. The method of the present invention can distinguish interstitial pneumonia involving DAD from other types of interstitial pneumonia (e.g., those involving OP) and predict the onset.

“Peripheral Blood Mononuclear Cells (PBMC)”

Mononuclear cells are a collective term for mononuclear mesenchymal cell groups widely distributed in connective tissues, blood, and lymphoid tissue in the whole body, and include macrophages in tissue, monocytes as precursor cells thereof, and lymphocytes. “Peripheral blood mononuclear cells (PBMC)” according to the present invention are mononuclear cells present in peripheral blood, and principally consist of monocytes and lymphocytes. Peripheral blood mononuclear cells can be isolated according to a known method or by using a commercially available kit or the like.

“Antitumor Cytotoxic Activity”

“Antitumor cytotoxic activity” means a function to cause death, functional disorder, or growth inhibition to tumor cells. NK cells exhibit high cytotoxicity to tumor cells via ligands on their surfaces, and γδ T cells exhibit high cytotoxicity to tumor cells having high intracellular IPP concentrations, and they produce cytokines such as IFN-γ and TNF-α, exhibiting anti-tumor cell activity. Known as “effector T cells” having tumor cell-damaging ability are αβ T cells, γδ T cells, and NK cells.

“αβ T Cells”

“αβ T cells” are T cells having a T cell receptor composed of two glycoproteins of an α chain and a β chain, and account for most of the peripheral blood lymphocytes. αβ T cells recognize antigenic peptide/MHC complex via a TCR/CD3 complex. Accordingly, information on antigenic peptides is required for analysis of αβ T cells. Currently, antigenic peptides that T cells recognize have been identified; however, multiple types of antigenic peptides are expected to be present even for one type of tumor, and it is difficult to understand the overview and analyze it.

“γδ T cells”

“γδ T cells” are T cells having a T cell receptor composed of two glycoproteins of a γ chain and a 6 chain on the cell surface. Normally, the number of γδ T cells is far smaller than that of αβ T cells. Approximately 4% γδ T cells are present in CD3-positive T cells in peripheral blood mononuclear cells, and 50 to 75% thereof are Vγ2Vδ2 T cells that express “Vγ2” (also referred to as Vγ9) and “Vδ2” in the TCR variable region (Vδ2⁺γδ T cells).

While almost no antigen molecules that activate γδ T cells are known, the present inventors have reported that synthetic alkyl phosphate such as monoethyl phosphate serves as an antigen for γδ T cells (Tanaka Y et al., PNAS USA 91:8175-8179, 1994), and that γδ T cells that have recognized a pyrophosphomonoester metabolite such as isopentenyl diphosphate (IPP) as an antigen and have been activated by IPP have strong antitumor activity (Tanaka et al., Nature, 375: 155-158, 1995). Further, the present inventors have reported that γδ T cells are activated when antigen-presenting cells (Miyagawa F et al., J. Immunol 166: 5508-5514, 2001) or tumor cells (Kato Y. et al., J. Immunol 167: 5092-5098, 2001) are treated with nitrogen-containing bisphosphonate. Although γδ T cells leave unresolved matters on details of their antigen recognition mechanism, it is possible to analyze it.

“Natural Killer (NK) Cells”

“NK cells” according to the present invention are lymphocytes belonging neither to T cells nor to B cells, and exhibit toxic activity to tumor cells, certain virus-infected cells, transplanted bone marrow cells, and so on without being restricted by major histocompatibility (MHC) antigens. On surfaces of NK cells, there exist an activation receptor that binds to a ligand on surfaces of target cells to induce cytotoxic activity, and a suppression receptor that recognizes self MHC class I molecules to suppress signals from the activation receptor. Thus, NK cells normally receive negative signals derived from MHC, and exhibit antitumor cytotoxicity when MHC on tumor cells is lost. However, examination on expression of PD-1 in human NK cells finds that it is difficult to confirm the expression of PD-1 and thus to analyze it.

The present inventors have found that combination of IL-2 and IL-18 enables efficient growth of NK cells (WO 2016/021720). NK cells can be identified by expression of CD56. NK cells after being stimulated with IL-2 and IL-18 are expressing HLA-DR, HLA-DQ, and CD80, which are associated with antigen-presenting cells, suggesting that NK cells not only destruct cancer cells but also activate the immune defense by presenting a cancer antigen to T cells.

“Killer T Cells (CTL)”

“Killer T cells (CTL)” are referred to as cytotoxic T lymphocytes (CTL), and are foreign matters for a host. CTLs recognize and disorder cells having an alloantigen or viral antigen. CTLs have a CD8 antigen and a T cell receptor consisting of an α chain and a β chain on the cell surface. “CD8-positive T cells” receive the presentation of an MHC-class I antigen and antigen peptide from antigen-presenting cells, and are activated to acquire cytotoxic activity. The activated CTLs disorder cells by releasing perforin, granzyme, or TNF or stimulating an Fas antigen of target cells to induce apoptosis.

2. Risk of Onset of Severe Interstitial Pneumonia Caused by Immune Checkpoint Inhibitor

By cancelling suppression of the functions of effector T cells due to tumor cells, immune checkpoint inhibitors recover their original functions. For cancer patients having extremely few effector T cells, however, even when the functions of effector cells are recovered, efficient antitumor effect would not be successfully obtained because of the insufficient absolute number of effector cells. The present invention is characterized by predicting the risk of onset of severe interstitial pneumonia caused by an immune checkpoint inhibitor by measuring the number (proportion) and functions of γδ T cells, which are effector cells.

2.1 Specimens (Samples)

Specimens (samples) to be used in the present invention are peripheral blood mononuclear cells isolated from subjects, that is, subjects who are considering use of an immune checkpoint inhibitor or already using it. The amount of peripheral blood required for one measurement is at least 10 ml, and preferably 10 ml to 20 ml.

Peripheral blood mononuclear cells can be isolated in such a manner that peripheral blood collected from a subject, to which an appropriate amount of an anticoagulant is added in advance, as necessary, is diluted with physiological buffer such as PBS according to a conventional method, and then subjected to density gradient centrifugation, specific gravity centrifugation, or the like. Mononuclear cells isolated are diluted with human T cell medium such as Yssel's medium, Iscov's medium, and RPMI1640 medium to adjust to a certain concentration, for example, 1×10⁴ cells/ml to 1×10⁷ cells/ml, preferably 5×10⁵ cells/ml to 3×10⁶ cells/ml.

2.2 Measurement Target

### (a) Cell Count or Proportion of Vδ2⁺γδ T Cells in Peripheral Blood Mononuclear Cells

The “cell count or proportion of Vδ2⁺γδ T cells in peripheral blood mononuclear cells” can be measured by using specific surface markers.

For example, T cells have a CD3 antigen on their surfaces, and γδ T cells further have a receptor consisting of two glycoproteins of a γ chain and a δ chain on their surfaces. Accordingly, use of an antibody that specifically binds to CD3 and an antibody that specifically binds to one or both of the γ chain and 6 chain (e.g., an anti-Vγ2 antibody, an anti-Vδ1 antibody, an anti-Vδ2 antibody) for peripheral blood mononuclear cells isolated from a subject enables measurement of the amount of γδ T cells in the peripheral blood mononuclear cells. Because most of the γδ T cells are Vγ2Vδ2 T cells (Vδ2⁺γδ T cells), as described above, Vγ2Vδ2 can be substantially detected by using Vδ2 as an index. That is, CD3⁺Vδ2⁺ cells (Vδ2⁺γδ T cells) detected by using an anti-Vδ2 antibody and a CD3 antigen, which is a T cell antigen, can be used for assessment as an indicator of the number or proportion of γδ T cells. The “cell count or proportion of Vδ2⁺γδ T cells in peripheral blood mononuclear cells” can be determined in a simple and quick manner by using flow cytometry or an image analyzer, described later, in measurement.

Specifically, the cell count or proportion is measured for a certain number of peripheral blood mononuclear cells (1×10⁷ peripheral blood mononuclear cells for a cutoff value shown later) after collection of peripheral blood (on Day 0). Since the immune condition of a subject may vary, it is preferred to perform collection of peripheral blood immediately before treatment.

(b) Cell Count or Proportion of Vδ2⁺γδ T Cells after Antigenic Stimulation in Peripheral Blood Mononuclear Cells

The “cell count or proportion of Vδ2⁺γδ T cells after antigenic stimulation in peripheral blood mononuclear cells” indicates the growth capacity of Vδ2⁺γδ T cells.

Any antigen may be used, without limitation, that is recognized by γδ T cells and capable of activating them. Applicable as such an antigen are peptide antigens such as IL-2, IL-7, IL-12, IL-15, IL-18, IL-21, IL-23, and interferon-γ; phosphomonoester compounds, triphosphomonoester compounds, tetraphosphomonoester compounds, triphosphodiester compounds, and tetraphosphodiester compounds including synthetic alkyl phosphates such as (E)-4-hydroxy-3-methyl-2-butenyl diphosphate (HMB-PP), which is produced by mycobacteria, plasmodia, and so on, 2-methyl-3-butenyl diphosphate (2M3BPP), and monoethyl phosphate; pyrophosphoric acid derivatives, as typified by pyrophosphomonoester compounds having a C1-5 alkyl group or salts thereof (e.g., a compound described in Japanese Patent Laid-Open No. 2003-128555) such as isopentenyl diphosphate (IPP), disodium monoethyl pyrophpsphate, disodium monomethyl pyrophosphate, and disodium monopropyl pyrophosphate; nitrogen-containing bisphosphonate compounds, as typified by bisphosphonate compounds obtained by introducing alkylamine or alkenylamine to the geminal carbon atom in nitrogen-containing bisphosphonate, or esters thereof, or salts of them (e.g., a compound described in WO 2016/098904 and WO 2016/125757 (such as PTA shown later)); non-peptide antigens such as alkylamines, alkyl alcohols, alkenyl alcohols, and isoprenyl alcohol; and human-derived tumor cells and peripheral blood-conditioned medium. The antigen may be a fragment thereof as long as the fragment functions.

To measure the cell count or proportion after antigenic stimulation, any of the listed antigens is added to culture solution containing isolated peripheral blood mononuclear cells, and after a certain period of time, measurement is performed in the same manner as in the above section (a). The amount of the antigen to be added is appropriately determined according to the γδ T cell activation capacity of the antigen to be used. The time until measurement after the addition of the antigen is determined according to the antigen to be used, similarly, and typically 0.5 hours or longer, and preferably about 12 hours to 14 days.

In the case of IL-2, for example, IL-2 is added, for example, to reach 10 to 1000 IU/ml, preferably to reach 20 to 200 IU/ml, incubation is performed in an atmosphere at 37° C. and 5% CO₂, and the cell count or proportion of 76 T cells is measured after 3 days to 14 days, preferably after 7 days to 11 days.

In the case of a pyrophosphomonoester derivative, the pyrophosphomonoester derivative is added, for example, to reach 10 pM to 500 μM, preferably to reach 100 pM to 100 μM, incubation is performed in an atmosphere at 37° C. and 5% CO₂, and the cell count or proportion of γδ T cells is measured after 3 days to 14 days, preferably after 7 days to 11 days.

In the case of a nitrogen-containing bisphosphonate derivative such as PTA, the nitrogen-containing bisphosphonate derivative is added, for example, to reach 1 nM to 500 μM, preferably to reach 10 nM to 5 μM (e.g., 1 μM PTA), incubation is performed in an atmosphere at 37° C. and 5% CO₂, and the cell count or proportion of γδ T cells is measured after 3 days to 14 days, preferably after 7 days to 11 days.

### (c) Cell Count or Proportion of Vδ2⁺γδ T Cells in Peripheral Blood T Cells

The “cell count or proportion of Vδ2⁺γδ T cells in peripheral blood T cells” can be determined by using an anti-CD3 antibody, which is specific to T cells, and an anti-Vδ2 antibody. In measurement, the determination can be made with use of flow cytometry or an image analyzer, described later.

(d) Cell Count or Proportion of Vδ2⁺γδ T Cells after Antigenic Stimulation in Peripheral Blood T Cells

The “cell count or proportion of Vδ2⁺γδ T cells after antigenic stimulation in peripheral blood T cells” indicates the growth capacity of Vδ2⁺γδ T cells. Antigenic stimulation and methods for measuring the cell count or proportion of Vδ2⁺γδ T cells after the antigenic stimulation can be performed according to the methods described in (b).

Although any of (a) to (d) above can be used as an index in the present invention, it is rather preferred to use peripheral blood T cells as a sample for subjects having many CD3⁻Vδ2⁻ cells, as described later.

While most of the γδ T cells are normally Vδ2⁺ cells in healthy individuals, Vδ1 may be abundant in cancer patients. Even for samples derived from such patients, antigenic stimulation allows Vδ2⁺ cells to grow and Vδ1 is diminished to become undetectable, and hence Vδ2⁺γδ T cells can be evaluated as an indicator of the number or proportion of γδ T cells. Therefore, use of the cell count or proportion of Vδ2⁺γδ T cells after antigenic stimulation in a sample is preferred for subjects having many Vδ1⁺γδ T cells.

2.3 Measurement of Cell Count or Proportion

Flow Cytometry

The cell count or proportion of Vδ2⁺γδ T cells in peripheral blood mononuclear cells or peripheral blood T cells can be measured through flow cytometry using an antibody specific to the surface antigen of each cell. Flow cytometry is a cell measurement method in which cells suspended in a fluid are introduced to a sensing zone one by one and fluorescence or scattering light is measured in the single stream, thereby being capable of quantitatively analyzing a large number of cells one by one in a short time.

The cell count or proportion of Vδ2⁺γδ T cells can be measured in a simple manner with a two-color fluorescence histogram, for example, using CD3, a T cell marker, and Vδ2, a γδ T cell marker. Specifically speaking, when peripheral blood mononuclear cells are analyzed with a two-color fluorescence histogram using a CD3 antibody and a Vδ2 antibody, CD3⁺Vδ2⁻ corresponds to ay T cells (G1) and CD3⁺Vδ2⁺ corresponds to γδ T cells (G2), and in addition to them cells of CD3⁻Vδ2 (G3) can be detected. G2/G1+G2+G3 corresponds to the proportion of Vδ2⁺γδ T cells in peripheral blood mononuclear cells, and G2/G1+G2 corresponds to the cell count and proportion of Vδ2⁺γδ T cells in peripheral blood T cells.

Image Cytometry (Image Analyzer)

The cell count or proportion of Vδ2⁺γδ T cells in peripheral blood mononuclear cells can be measured through image cytometry using an antibody specific to the surface antigen of each cell. Image cytometry is a cell measurement method in which cells on a multi-well plate or microscope slide are scanned with a laser to acquire their fluorescence image or scattering light/transmitted light image and the image is subjected to image processing, thereby being capable of quantitatively analyzing a large number of cells one by one in a short time.

As with the case of flow cytometry, the cell count or proportion of Vδ2⁺γδ T cells in peripheral blood mononuclear cells or peripheral blood T cells can be measured in a simple manner with a scattering light image or two-color fluorescence image using CD3, a T cell marker, and Vδ2.

2.4 Prediction/Assessment Method

### (1) Prediction Based on Number/Proportion of Vδ2⁺γδ T Cells in Peripheral Blood Mononuclear Cells

If the cell count or proportion of Vδ2⁺γδ T cells in peripheral blood mononuclear cells in (a) and/or the cell count or proportion of Vδ2⁺γδ T cells after antigenic stimulation in peripheral blood mononuclear cells in (b) are/is equal to or more than a specific cutoff value, the risk of onset of severe interstitial pneumonia caused by an immune checkpoint inhibitor can be predicted to be high. Further, whether treatment with the immune checkpoint inhibitor would be appropriate or not can be assessed based on the risk of onset.

The cutoff value is appropriately determined according to the immune checkpoint inhibitor to be used and the number of and cell culture period for peripheral blood mononuclear cells.

Assuming that the number of peripheral blood mononuclear cells is 1×10⁷, the cutoff value for the cell count in (a) is typically in the range of 0.5×10⁵ to 15×10⁵, preferably in the range of 0.5×10⁵ to 14×10⁵, 0.5×10⁵ to 13×10⁵, 0.5×10⁵ to 12×10⁵, 0.5×10⁵ to 11×10⁵, 0.5×10⁵ to 10×10⁵, 0.5×10⁵ to 9×10⁵, 0.5×10⁵ to 8×10⁵, 0.5×10⁵ to 7×10⁵, 0.5×10⁵ to 6×10⁵, or 0.5×10⁵ to 5×10⁵, more preferably in the range of 1×10⁵ to 15×10⁵, 1×10⁵ to 14×10⁵, 1×10⁵ to 13×10⁵, 1×10⁵ to 12×10⁵, 1×10⁵ to 11×10⁵, 1×10⁵ to 10×10⁵, 1×10⁵ to 9×10⁵, 1×10⁵ to 8×10⁵, 1×10⁵ to 7×10⁵, 1×10⁵ to 6×10⁵, 1×10⁵ to 5×10⁵, or 1×10⁵ to 4×10⁵, and particularly preferably in the range of 1×10⁵ to 3×10⁵.

The cutoff value for the cell proportion is typically in the range of 0.5 to 15%, preferably in the range of 0.5 to 14%, 0.5 to 13%, 0.5 to 12%, 0.5 to 11%, 0.5 to 10%, 0.5 to 9%, 0.5 to 8%, 0.5 to 7%, 0.5 to 6%, 0.5 to 5%, 0.6 to 5%, 0.7 to 5%, 0.8 to 5%, 0.9 to 5%, 1.0 to 5%, 0.6 to 4%, 0.7 to 4%, 0.8 to 4%, 0.9 to 4%, or 1.0 to 4%, more preferably in the range of 0.6 to 3%, 0.7 to 3%, 0.8 to 3%, or 0.9 to 3%, and particularly preferably in the range of 1 to 3%.

The cutoff value for the cell count in the case that antigenic stimulation is applied in (b) depends on the antigenic stimulation to be applied, and amounts to more than several tens of times the above value, and preferably amounts to 100 to 2000 times the above value. In the case of antigenic stimulation using PTA and IL-2, for example, the cell count increases by 200 to 3000 times and the cell proportion reaches more than 98%. Vδ2⁺γδ T cells have high reactivity in subjects having a high risk of onset of severe interstitial pneumonia, and the cells aggregate when antigenic stimulation with a pyrophosphoric acid derivative or a bisphosphonate compound is applied, which allows assessment by visual observation. For example, peripheral blood mononuclear cells are subjected to the action of 1 μM PTA, and cell aggregation on Day 1 is assessed by visual observation.

### (2) Prediction of Number/Proportion of Vδ2⁺ T Cells in Peripheral Blood T Cells

If the cell count or proportion of Vδ2⁺γδ T cells in peripheral blood T cells in (c) and/or the cell count or proportion of Vδ2⁺γδ T cells after antigenic stimulation in peripheral blood T cells in (d) are/is equal to or more than a specific cutoff value, the risk of onset of severe interstitial pneumonia caused by an immune checkpoint inhibitor can be predicted to be high. Further, whether treatment with the immune checkpoint inhibitor would be appropriate or not can be assessed based on the risk of onset.

In general, the response of a subject can be predicted with the above-described cell count or proportion of Vδ2⁺γδ T cells in peripheral blood mononuclear cells; however, for subjects having many cells of CD3⁻Vδ2⁻ (G3), it is rather preferred to make diagnosis targeting the cell count or proportion of Vδ2⁺γδ T cells in peripheral blood T cells excluding cells of CD3⁻Vδ2⁻.

The cutoff value is appropriately determined according to the immune checkpoint inhibitor to be used and the number of and cell culture period for peripheral blood T cells.

Assuming that the number of T cells is 1×10⁷, the cutoff value for the cell count in (c) is typically in the range of 1×10⁵ to 20×10⁵, preferably in the range of 1×10⁵ to 19×10⁵, 1×10⁵ to 18×10⁵, 1×10⁵ to 17×10⁵, 1×10⁵ to 16×10⁵, 1×10⁵ to 15×10⁵, 1×10⁵ to 14×10⁵, 1×10⁵ to 13×10⁵, 1×10⁵ to 12×10⁵, 1×10⁵ to 11×10⁵, 1×10⁵ to 10×10⁵, 1×10⁵ to 9×10⁵, 1×10⁵ to 8×10⁵, 1×10⁵ to 7×10⁵, 1×10⁵ to 6×10⁵, or 1×10⁵ to 5×10⁵, more preferably in the range of 2×10⁵ to 5×10⁵ or 2×10⁵ to 4×10⁵, and particularly preferably in the range of 2×10⁵ to 3×10⁵.

The cutoff value for the cell proportion is typically in the range of 1 to 20%, preferably in the range of 1 to 19%, 1 to 18%, 1 to 17%, 1 to 16%, 1 to 15%, 1 to 14%, 1 to 13%, 1 to 12%, 1 to 11%, 1 to 10%, 1 to 9%, 1 to 8%, 1 to 7%, 1 to 6%, 1 to 5%, 1 to 4%, or 1 to 3%, more preferably in the range of 2 to 5% or 2 to 4%, and particularly preferably in the range of 2 to 3%.

The cutoff value for the cell count in the case that antigenic stimulation is applied in (d) depends on the antigenic stimulation to be applied, and amounts to more than several tens of times the above value, and preferably amounts to 100 to 2000 times the above value. In the case of antigenic stimulation using PTA and IL-2, for example, the cell count increases by 200 to 3000 times and the cell proportion reaches more than 98%.

Vδ2⁺γδ T cells have high reactivity in subjects having a high risk of onset of severe interstitial pneumonia, and the cells aggregate when antigenic stimulation with a pyrophosphoric acid derivative or a bisphosphonate compound is applied, which allows assessment by visual observation. For example, peripheral blood mononuclear cells are subjected to the action of 1 μM PTA, and cell aggregation on Day 1 is assessed by visual observation.

2.5 Other Methods

In addition to the methods described above, whether treatment would be appropriate or not may be assessed by combining the following indexes (e) to (i) according to the immune checkpoint inhibitor to be used or therapeutic purpose.

(e) The expression level of PD-1 in γδ T cells after antigenic stimulation
(f) The antitumor cytotoxic activity of γδ T cells after antigenic stimulation
(g) The cell count or proportion of NK cells in peripheral blood mononuclear cells isolated from a subject
(h) The cell count or proportion of NK cells in peripheral blood mononuclear cells isolated from a subject after growth stimulation
(i) The antitumor cytotoxic activity of NK cells after the growth stimulation

As described above, the indexes may be determined with assuming γδ T cells as Vδ2⁺γδ T cells.

(e) Expression Level of PD-1 in γδ T Cells after Antigenic Stimulation

The “expression level of PD-1 in γδ T cells after antigenic stimulation” is an index of responsivity to an immune checkpoint inhibitor. Thus, more precise treatment can be achieved through evaluation of responsivity to an immune checkpoint inhibitor in combination with the risk of onset of severe interstitial pneumonia.

Any antigen may be used, without limitation, that is recognized by γδ T cells and capable of activating them, and the antigens listed in (b) above can be used. The amount of the antigen to be added and the time until measurement after the addition of the antigen are also as described above in (b). The expression level of PD-1 can be quantitatively analyzed in a simple manner by using the above-described flow cytometry or image cytometry.

(f) Antitumor Cytotoxic Activity of γδ T Cells after Antigenic Stimulation

The “antitumor cytotoxic activity of γδ T cells after antigenic stimulation” is an indicator of whether γδ T cells actually exert antitumor cytotoxic activity in response to an immune checkpoint inhibitor. Thus, more precise treatment can be achieved through evaluation of antitumor cytotoxic activity of γδ T cells in response to an immune checkpoint inhibitor in combination with the risk of onset of severe interstitial pneumonia.

Any antigen may be used, without limitation, that is recognized by γδ T cells and capable of activating them, and the antigens listed in (b) above can be used. The amount of the antigen to be added and the time until measurement after the addition of the antigen are also as described above in (b).

For common tumor cells, it is preferred for measurement of cytotoxic activity to stimulate with nitrogen-containing bisphosphonate (N-BP) or the like in order to enhance the antitumor cytotoxicity of γδ T cells. For example, tumor cells are first treated with N-BP, and a terpyridine derivative is pulsed 15 minutes before the completion of the treatment. The cancer cells are washed, and then subjected to the action of γδ T cells to evoke cytotoxicity. After 40 minutes, antitumor cytotoxic activity is measured with a method described later.

Also available is a method using tumor cells that are susceptible to the cytotoxicity of γδ T cells. For example, Daudi Burkitt lymphoma cells are affected by the cytotoxicity of γδ T cells even without being stimulated with N-BP. If Daudi cells are forced to express PD-L1, the effect of an anti-PD-L1 antibody can be measured in a simpler manner. Specifically, if being subjected to the action of γδ T cells, Daudi/PD-L1 cells undergo immunosuppression through PD-1/PD-L1 interaction. If an anti-PD-L1 antibody is added thereto, however, the PD-1/PD-L1 interaction is blocked to result in enhanced cytotoxicity. By using this system, the antitumor cytotoxic activity of an immune checkpoint inhibitor can be evaluated with ease in vitro.

Antitumor cytotoxic activity can be measured through a known method with use of a cultured cancer cell line, such as a β radioactivity measurement method, a γ radioactivity measurement method, a lactate dehydrogenase (LDH) activity measurement method, a time-resolved fluorescence method, and a non-RI system cytotoxicity measurement method (WO 2015/152111).

β Radioactivity Measurement Method

Target cells (tumor cells) are labeled with ³H-Proline and subjected to mixed culture together with effector cells (γδ T cells or NK cells), and the amount of ³H-Prolin (β radiation) emitted from the target cells through cell disorder caused by the effector cells is measured. The mixing ratio between target cells and effector cells (E/T ratio) and culture time are appropriately set according to the cells to be used, and the E/T ratio is adjusted, for example, to about E/T ratio=0.5 to 2.

Cytotoxic activity (%) represented by the following expression is calculated to evaluate cytotoxic activity.

Cytotoxic activity (%)=(E/T ratio)−Emission from target cells only/Emission when target cells are all disordered−Emission from target cells only

γ Radioactivity Measurement Method

Target cells (tumor cells) are labeled with ⁵¹Cr and subjected to mixed culture together with effector cells (γδ T cells or NK cells), and the amount of ³H-Prolin (γ radiation) emitted from the target cells through cell disorder caused by the effector cells is measured. In the same manner as for the β radioactivity measurement method, the mixing ratio between target cells and effector cells (E/T ratio) and culture time are appropriately set according to the cells to be used, and cytotoxic activity is evaluated through calculation of cytotoxic activity (%)

Time-Resolved Fluorescence Method

Target cells (tumor cells) are labeled with europium (Eu) and subjected to mixed culture together with effector cells (γδ T cells or NK cells), and the amount of ³Eu (fluorescence) emitted from the target cells through cell disorder caused by the effector cells is measured. In the same manner as for the β radioactivity measurement method and γ radioactivity measurement method, the mixing ratio between target cells and effector cells (E/T ratio) and culture time are appropriately set according to the cells to be used, and cytotoxic activity is evaluated through calculation of cytotoxic activity (%).

Lactate Dehydrogenase (LDH) Activity Measurement Method

Lactate dehydrogenase (LDH) is an enzyme present in the cytoplasm, and released into medium when a cell is disordered. The LDH released is quantified through formazan dye (absorbance at 490 nm) that is generated by reacting NADH, which is generated by lactate dehydrogenation reaction catalyzed by LDH, with ITN (tetrazolium salt). The method is highly safe because RI is not used. In the same manner as for the other methods, the mixing ratio between target cells and effector cells (E/T ratio) and culture time are appropriately set according to the cells to be used, and cytotoxic activity is evaluated through calculation of cytotoxic activity (%).

Non-RI Measurement Method Using Chelate Precursor

In the quick cytotoxic capacity measurement method with a non-RI system developed by the present inventors, tumor cells are treated with a chelating agent precursor. Specifically, tumor cells are first treated with a terpyridine derivative protected with a butanoyloxymethyl group. Then, the terpyridine derivative is incorporated in the cells because of its lipophilicity, and hydrolyzed by esterase, and a chelating agent having negative charge is accumulated in the cells. At this time, if being subjected to the action of immune effector cells, the tumor cells are disordered and their membrane structures are slightly destroyed. Then, the chelating agent quickly leaks into the culture supernatant. At this time, if a portion of the culture supernatant is collected and europium, a lanthanoid series metal, is added thereto, a chelate is formed and emits time-resolved fluorescence when being irradiated with excitation light. Through measurement of this time-resolved fluorescence, cytotoxicity can be quantified in a non-RI manner.

Time-resolved fluorescence is advantageous in that as compared with common fluorescent compounds, which emit fluorescence only for about 2 μsec after being irradiated with excitation light, fluorescence is emitted for a long time of about 100 μsec, which results in a larger difference from the background to lead to higher reliability of measurement. Among alkoxymethyl derivatives of terpyridine dicarboxylate to be used in the present assay method, the following compound can achieve a high maximum labeling level and a natural leakage rate of 20% or lower in most tumor cell lines.

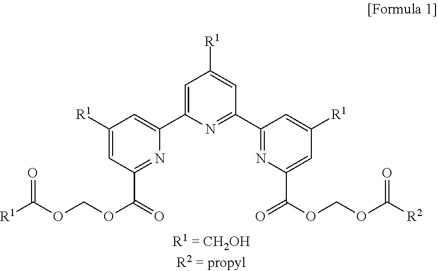

The tumor cell line to be used for measurement of antitumor cytotoxic activity is not limited. Examples of tumor cells include, but are not limited to, the human myeloid tumor or leukemia cell lines K562, HL60, EB1, CCRF-CEM, HEL-92.1.7, HSB, Jurkat, HuT-78, KG-1A, HNT-34, MOLT-4, MV4-11, NB-4, REH, RPMI-1788, TF-1, THP-1, TK6, and U937; the human lung cancer cell lines A-427, A-549, Calu-1, Calu-6, CLS-54, DMS-79, GCT, HEL-299, H-Messo-1, H-Messo-1A, LCLC-97TM1, LX-1, LX-289, MRC-5, MSTO-211H, NCI-H146, NCI-H209, NCI-H69, NCI-H82, NCI-H128, SCLC-21H, SCLC-22H, SK-LU-1, SK-MES-1, and SV-80; the human liver cancer cell lines Chang-Liver, Hep-G2, HuH-7, PLC-PRF-5, and SK-HEP-1; the human breast cancer cell lines BT-20, BT-474, BT-549, COLO-824, HBL-100, MA-CLS-2, MCF-7, MDA-MB-231, MX-1, SK-BR-3, T-47D, and ZR-75-1; the human ovarian cancer cell line HEY; the human gastric cancer cell lines AGS, CLS-145, HGC-27, MKN1, MKN28, and KATO-III; the human pancreatic cancer cell lines AsPC-1, Capan-1, Capan-2, DAN-G, FAMPAC, FAMPAC-A, PA-CLS52, and Panc-1; the human kidney cancer cell lines 293 (HEK-293), 769-P, 786-0, A-498, A-704, ACHN, CaKi-2, RC-124, RC-131, RC-134, RC-138, RC-142, RCC-AB (KTCTL-21), RCC-ER (KTCTL-13), RCC-EK (KTCTL-135), RCC-EW (KTCTL-2), RCC-AL4, RCC-FG1 (KTCTL-26), RCC-FG2 (KTCTL-26A), RCC-GH, RCC-GS (KTCTL-185), RCC-HB (KTCTL-48), RCC-JW (KTCTL-195), RCC-KL, RCC-KP (KTCTL-53), RCC-LR (KTCTL-120), RCC-MF (KTCTL-1M), RCC-MH (KTCTL-129), RCC-OF1 (KTCTL-54), RCC-GW, RCC-PR, RCC-WK (KTCTL-87), SK-NEP-1, and WT-CLS1; the human osteosarcoma cell lines CADO-ES1, HOS (TE-85), KHOS-240S, KHOS-312H, KHOS-NP, MG-63, MHH-ES1, MNNG-HOS, RD-ES, SaOS-2, SK-ES-1, SW-1353, TM-791, and U-20S; the human colorectal cancer cell lines CW2, DLD-1, and Colo320; the human malignant melanoma cell lines C32TG and G361; and the human prostate cancer cell lines PC-3, DU-145, and LNCaP. For NK cells, in particular, K562 cells, which are used as a standard cell line for antitumor cytotoxic activity test for NK cells, are preferred, and U937 histocyte-derived leukemia cells are preferred for γδ T cells.

To increase the objective response rate of an immune checkpoint inhibitor, development of a combination therapy with another immunotherapy is under way. For example, use of nivolumab (human-type anti-PD-1 monoclonal antibody) and ipilimumab (human-type anti-CTLA-4 monoclonal antibody) in combination has been reported to achieve an objective response rate of 60%. The present inventors have reported that use of IL-18 in combination with an anti-PD-1 antibody, anti-PD-L1 antibody, anti-PD-1 antibody, anti-PD-L1 antibody, or anti-CDLA-4 antibody is found to provide a synergistically enhanced antitumor effect (WO 2010/001617). In predicting the objective response rate of an immune checkpoint inhibitor in such a combination therapy, the antitumor cytotoxic activity may be examined in the presence of an antibody to be used in combination or cytokine.

### (g) Cell Count or Proportion of NK Cells in Peripheral Blood Mononuclear Cells

The “cell count or proportion of NK cells in peripheral blood mononuclear cells” can be measured by using a surface marker specific to peripheral blood mononuclear cells and that specific to NK cells. As described above, NK cells have antitumor cytotoxic activity. Accordingly, more precise treatment can be achieved evaluation of the number or proportion of NK cells in combination with the risk of onset of severe interstitial pneumonia.

NK cells have a CD56 antigen on their surfaces. Accordingly, the amount of NK cells in peripheral blood mononuclear cells can be measured by using an anti-CD3 antibody and anti-CD56 antibody. The “cell count or proportion of NK cells in peripheral blood mononuclear cells” can be determined in a simple and quick manner by using flow cytometry or an image analyzer, in measurement.

(h) Cell Count or Proportion of NK Cells in Peripheral Blood Mononuclear Cells after Growth Stimulation

The “cell count or proportion of NK cells in peripheral blood mononuclear cells after growth stimulation” indicates the growth capacity of NK cells. Accordingly, more precise treatment can be achieved through evaluation of the growth capacity of NK cells, which have antitumor cytotoxic activity, in combination with the risk of onset of severe interstitial pneumonia.

Any growth stimulation factor capable of stimulating the growth of NK cells may be used, without limitation. Examples thereof include IL-2, IL-7, IL-12, IL-15, IL-18, IL-21, IL-23, interferon-γ, and peripheral blood-conditioned medium. The growth stimulation factor may be a fragment thereof as long as the fragment functions.

To measure the cell count or proportion after growth stimulation, the growth stimulation factor is added to culture solution containing isolated peripheral blood mononuclear cells, and after a certain period of time, measurement is performed in the same manner as in the above section (b). The amount of the growth stimulation factor to be added is appropriately determined according to the growth stimulation capability of the growth stimulation factor to be used for NK cells. The time until measurement after the addition of the growth stimulation factor is appropriately determined according to the growth stimulation factor to be used, similarly, and typically 0.5 hours or longer, and preferably about 12 hours to 14 days.

In the case of IL-2, for example, IL-2 is added, for example, to reach 10 to 1000 IU/ml, preferably to reach 20 to 200 IU/ml, incubation is performed in an atmosphere at 37° C. and 5% CO₂, and the cell count or proportion of NK cells is measured after 3 days to 14 days, preferably after 7 days to 11 days.

In the case of interferon-γ, for example, interferon-γ is added, for example, to reach 1 to 10000 IU/ml, preferably to reach 10 to 1000 IU/ml, incubation is performed in an atmosphere at 37° C. and 5% CO₂, and the cell count or proportion of NK cells is measured after 1 day to 14 days, preferably after 3 days to 10 days.

In the case of IL-18, for example, IL-18 is added, for example, to reach 1 to 1000 IU/ml, preferably to reach 20 to 300 IU/ml, incubation is performed in an atmosphere at 37° C. and 5% CO₂, and the cell count or proportion of NK cells is measured after 1 day to 14 days, preferably after 3 days to 10 days.

(i) Antitumor Cytotoxic Activity of NK Cells after Growth Stimulation

The “antitumor cytotoxic activity of NK cells after growth stimulation” is an indicator of whether NK cells actually exert antitumor cytotoxic activity in response to an immune checkpoint inhibitor. Thus, more precise treatment can be achieved through evaluation of antitumor cytotoxic activity of NK cells in combination with the risk of onset of severe interstitial pneumonia.

Any antigen capable of stimulating the growth of NK cells may be used, without limitation, and any of the growth stimulation factors listed in (h) above can be used. The amount of the growth stimulation factor to be added and the time until measurement after the addition of the growth stimulation factor are also as described in (h) above.

Antitumor cytotoxic activity can be measured through a known method with use of a cultured cancer cell line, such as a β radioactivity measurement method, a γ radioactivity measurement method, a lactate dehydrogenase (LDH) activity measurement method, a time-resolved fluorescence method, and a non-RI system cytotoxicity measurement method (WO 2015/152111).

3. Reagents/Kit for Diagnosis

The present invention further provides reagents and a kit for the above-mentioned prediction of the effect of an immune checkpoint inhibitor.

The kit of the present invention includes (i) an anti-CD3 antibody and (ii) an anti-Vδ2 antibody as essential components, and may include an instruction for assessment (diagnosis).

The kit of the present invention may further include (iii) a pyrophosphomonoester derivative or a nitrogen-containing bisphosphonate derivative, and/or (iv) IL-18.

In the kit of the present invention, each antibody may be appropriately labeled or immobilized. Each antibody may be an antibody fragment thereof as long as the antibody fragment is applicable to detection of antigen molecules of interest. Examples of antibody fragments include F(ab′)₂, Fab′, Fab, Fv, scFv, rIgG, and Fc.

In addition to the above-mentioned components, the kit of the present invention includes various reagents (e.g., an anti-CD4 antibody, an anti-CD8 antibody), a secondary antibody, substrate solution, a tumor cell line (e.g., a K562 cell line), medium (e.g., Yessel' medium), and so on, required for the above-described flow cytometry or image cytometry and measurement of antitumor cytotoxic activity. In addition, (i) to (iv) above and other components may be each provided individually as a reagent for assessment (diagnosis).

4. Companion Diagnostics and Therapeutic Strategy

The method for predicting the risk of onset of severe interstitial pneumonia caused by an immune checkpoint inhibitor, method for assessing (diagnostic method) whether treatment with the immune checkpoint inhibitor would be appropriate or not according to the method, reagents (diagnostic agents), and kit (kit for diagnosis) in the present invention can be used for clinical examination to predict the effect and adverse effect of an immune checkpoint inhibitor before administration, what is called companion diagnostics.

Examples of immune checkpoint inhibitors targeted are as described above, and include, but are not limited to, the anti-PD-1 antibodies nivolumab (Opdivo) and pembrolizumab (MK-3475); the anti-PD-L1 antibodies pidilizumab (CT-011), MPDL3280A/RG-7446, MEDI4736, MSB0010718C, and MED10680/AMP-514; an anti-PD-L2 antibody; the anti-CTLA-4 antibodies ipilimumab (MDX-010) and tremelimumab (CP675, 206); the anti-killer cell immunoglobulin-like receptor (KRI) antibody lirilumab (IPH2102/BMS-986015); the anti-CD137 antibodies urelumab (BMS-663513) and PF-05082566; the anti-LAG3 antibody BMS-986016; and the anti-OX40 antibody MEDI6469.

Through prediction of the risk of onset of severe interstitial pneumonia caused by an immune checkpoint inhibitor by using the prediction method (diagnostic method), reagents (diagnostic agents), and kit (kit for diagnosis) of the present invention, whether administration to a subject (patient) would be appropriate or not is determined; then, the immune checkpoint inhibitor is administered based on the result; thus, a series of therapeutic strategies with an immune checkpoint inhibitor is provided. Such a treatment method with an immune checkpoint inhibitor is also included in the present invention.

Diseases targeted by the treatment method are diseases that can be targeted by immune checkpoint inhibitors (such as cancer, infections). Examples of cancer include bone cancer, pancreatic cancer, skin cancer, head-and-neck cancer, melanoma, uterine cancer, ovarian cancer, rectal cancer, anal cancer, gastric cancer, testis cancer, uterine cancer, fallopian tube carcinoma, endometrial carcinoma, cervix carcinoma, vaginal carcinoma, vulval carcinoma, Hodgkin's disease, non-Hodgkin's lymphoma, esophageal carcinoma, small intestine cancer, endocrine system cancer, thyroid cancer, parathyroid cancer, adrenal gland cancer, parenchymal sarcoma, urethral cancer, penis cancer, acute myeloid leukemia, chronic myeloid leukemia, acute lymphoblastic leukemia, chronic leukemia, acute leukemia, childhood solid cancer, lymphocytic lymphoma, bladder cancer, kidney cancer, ureteric cancer, renal pelvis carcinoma, central nervous system (CNS) tumor, primary CNS lymphoma, tumor vasculogenesis, spinal tumor, brain stem glioma, pituitary adenoma, Kaposi's sarcoma, squamous cell carcinoma, planocellular carcinoma, T-cell lymphoma, and environmentally induced tumor. In particular, the treatment method is preferably applicable to metastatic cancer and lung cancer, which involve expression of PD-L1.

Examples of infections can include HIV infection (AIDS), hepatitis, herpes, malaria, dengue fever, leishmaniasis, flu, dysentery, pneumonia, tuberculosis, sepsis, and listeriosis. In particular, the treatment method is preferably applicable to HIV infection, which causes serious immunodeficiency.

In addition to the above diseases, the treatment method can be used for diagnosis on whether administration would be appropriate or not in Alzheimer-type dementia (Kuti Baruch1, et al. Nature Medicine. 2016; 22(2): 135-7), cerebral amyloid angiopathy, Down's syndrome, age-related macular degeneration, dementia with Lewy body, Parkinson's disease, multiple system atrophy, tauopathy, frontotemporal lobar degeneration, argyrophilic grain dementia, amyotrophic lateral sclerosis, diabetes mellitus, amyotrophic lateral sclerosis (ALS), and so on.

5. Pharmaceutical Composition Containing Immune Checkpoint Inhibitor Targeting Subjects Having a Low Risk of Onset of Severe Interstitial Pneumonia

The present invention provides a novel application of an immune checkpoint inhibitor targeting subjects assessed to have a low risk of onset of severe interstitial pneumonia caused by the immune checkpoint inhibitor through prediction of the risk of onset. The present invention further provides a pharmaceutical composition containing such an immune checkpoint inhibitor and characterized in that the pharmaceutical composition is used for treating or preventing tumor with suppression of the onset of severe interstitial pneumonia, and used for subjects assessed with the above-described method to have a low risk of onset of severe interstitial pneumonia.

6. Others

The method and reagents/kit of the present invention can be used not only for diagnosis before use on whether administration of an immune checkpoint inhibitor would be appropriate or not, but also for risk prediction after initiation of treatment, and diagnosis on whether in treatment with an immune checkpoint inhibitor, administration thereof would be appropriate or not in viral infections such as HIV infection, protozoan infections, bacterial infections, and so on.

### EXAMPLES

The present invention will be specifically described with reference to Examples; however, the present invention is not limited to these Examples.

### Example 1: Comparison of γδ T Cells and NK Cells in Peripheral Blood of Healthy Individuals and Lung Cancer Patients

What matters in cancer immunotherapy using an immune checkpoint inhibitor is immune condition including the number of T cells, which are effector cells, and expression of PD-1. In an extreme argument, administration of an immune checkpoint inhibitor would be ineffective with respect to antitumor cytotoxicity for cases of cancer patients in which the immune system is exhausted and there are almost no or extremely few antitumor cytotoxic T cells.

Assuming that “if tumor cells cause the PD-1 immune checkpoint to be involved in inducing immunotolerance to tumor-specific immune effector cells, the mechanism of action of αβ T cells and that of γδ T cells are the same”, induction of immunotolerance to αβ T cells and induction of immunotolerance to γδ T cells are inferred to occur simultaneously. It follows that if the state of immunotolerance of γδ T cells is successfully assessed, the state of immunotolerance of αβ T cells can be inferred, too. In view of this, in this Example, the number of γδ T cells of Vγ2Vδ2 and antigen reactivity thereof in healthy individuals and cancer patients were examined as validation of the hypothesis (it should be noted that half or more of the γδ T cells are of Vγ2Vδ2 in healthy adults, and if Vδ2 is used as an index, Vγ2Vδ2 is substantially detected).

1. Materials and Methods

### (1) Numbers or Proportions of Vγ2Vδ2 T Cells and NK Cells

The number or proportion of γδ T cells in peripheral blood mononuclear cells (PBMCs) is analyzed through two-color flow cytometry according to the following procedure.

Peripheral blood (10 ml) is collected from each lung cancer patient, and a PBMC fraction is prepared according to a conventional method and suspended in 50 μl of PBS/2% FCS. To each well, antibodies each in 3 μl are added, and the wells are left to stand on ice for 30 minutes, washed three times with 2% FCS/PBS, and analyzed with flow cytometry (FACSCalibur™, BD Biosciences).

The number and proportion of γδ T cells (Vδ2-positive cells) in PMBCs can be determined through two-color flow cytometry using an anti-CD3 antibody and an anti-Vδ2 antibody.

The number and proportion of NK cells (CD56-positive cells) in PMBCs can be determined through two-color flow cytometry using an anti-CD3 antibody and an anti-CD56 antibody.

CD3 is a surface marker of T cells, and CD56 is a surface marker of NK cells. Here, Vδ2 is used for detection of γδ T cells. The reason is as follows: most of the γδ T cells in healthy individuals are Vδ2-positive cells; even in a sample with many Vδ1-positive cells, Vδ2-positive cells grow and Vδ1-positive cells become undetectable after antigenic stimulation described later, and hence Vδ2-positive cells can be evaluated as γδ T cells.

### (2) Antigenic Stimulation (Growth Induction) of Vγ2Vδ2 T Cells

To PMBCs (3 ml), 3 μl of 1 mM PTA stock solution (in DMSO) is added to a final concentration of 1 μM. A suspension of Vδ2-positive cells is transferred to two wells (1.5 ml/well, two wells) of a 24-well plate. IL-2 and IL-18 are added to the two wells to final concentrations of 100 U/ml and 100 ng/ml, respectively, and incubation is performed at 37° C. and 5% CO₂. From Day 1, IL-2 or IL-2/IL-18 is added every day to the medium of PBMCs.

*PTA is a nitrogen-containing bisphosphonate having the following structure (WO 2016/125757, and Medicinal Chemistry, 2007, 85-99), and activates γδ T cells by inhibiting FPPS synthesis.

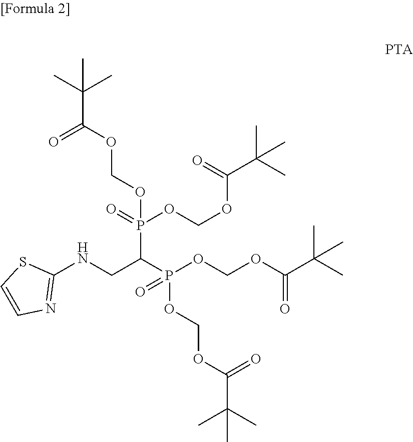 [SMILES: CC(C)(C)C(=O)OCOP(=O)(OCOC(=O)C(C)(C)C)C(CNc1nccs1)P(=O)(OCOC(=O)C(C)(C)C)OCOC(=O)C(C)(C)C]

Stimulation with Zol (Zometa)/IL-2/IL-18 is performed in the same manner except that Zol (1 μM) is used in place of PTA (1 μM).

(3) Number or Proportion of Vγ2Vδ2 T Cells after Antigenic Stimulation

Cells are collected on Day 11, and the number or proportion of γS T cells is analyzed through two-color flow cytometry.

(4) Stimulation of NK Cells with IL-2/IL-18

NK cells are purified from PMBCs according to a conventional method using an anti-CD3 antibody labeled with MACS® Beads. Specifically, PMBCs (3 ml) are transferred into a 15-ml conical tube, and centrifuged at 1700 rpm and 4° C. for 5 minutes. Subsequently, the supernatant is removed by suction, and cell pellets are dispersed and resuspended in 80 μl of PBS/0.5% BSA/2 mM EDTA. To the cell suspension, 20 μl of an anti-CD3 antibody labeled with MACS® Beads (Mylteny Biotec) is added, and the cell suspension is incubated at 4° C. for 15 minutes. After 15 minutes, 2 ml of PBS/0.5% BSA/2 mM EDTA is added to resuspend the cells. Subsequently, centrifugation is performed at 300×g and 4° C. for 10 minutes, and the supernatant is removed by suction. The cell pellets are dispersed and resuspended in 1 ml of PBS/0.5% BSA/2 mM EDTA. The cell suspension is applied to an LD column (composed of magnetic spheres) equilibrated with PBS/0.5% BSA/2 mM EDTA. CD3-negative cells are eluted twice with 1 ml of PBS/0.5% BSA/2 mM EDTA. Centrifugation is performed at 1700 rpm and 4° C. for 5 minutes, and the supernatant is discarded and the residual supernatant is removed by suction with an aspirator. Subsequently, the cell pellets are dispersed, and the CD3-negative cells are resuspended in 1.5 ml of YM-AB medium.

The suspension of NK cells is transferred to a 24-well plate (1.5 ml/well, one well), IL-2 and IL-18 are added to the well to final concentrations of 100 IU/ml and 100 ng/ml, respectively, and incubation is performed at 37° C. and 5% CO₂. From Day 0, IL-2/IL-18 is added every day to the medium for 10 days.

(5) Number or Proportion of NK Cells after Growth Stimulation

Cells are collected on Day 11, and the number or proportion of NK cells is analyzed through two-color flow cytometry according to (1).

2. Results

### (1) Proportion of γδ T Cells in Healthy Individuals

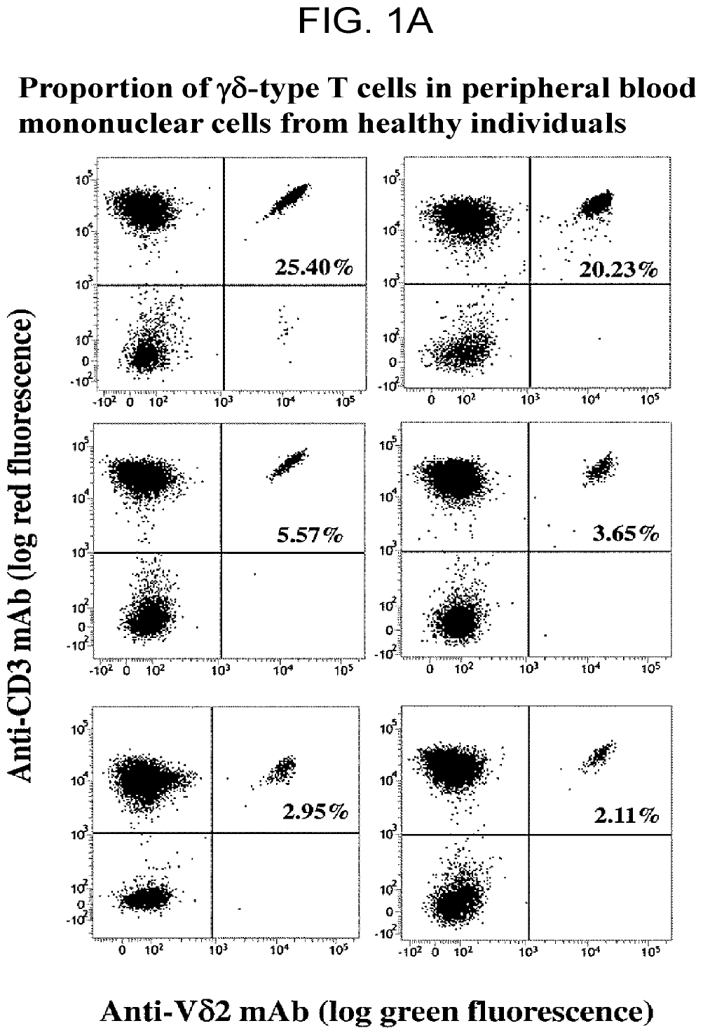

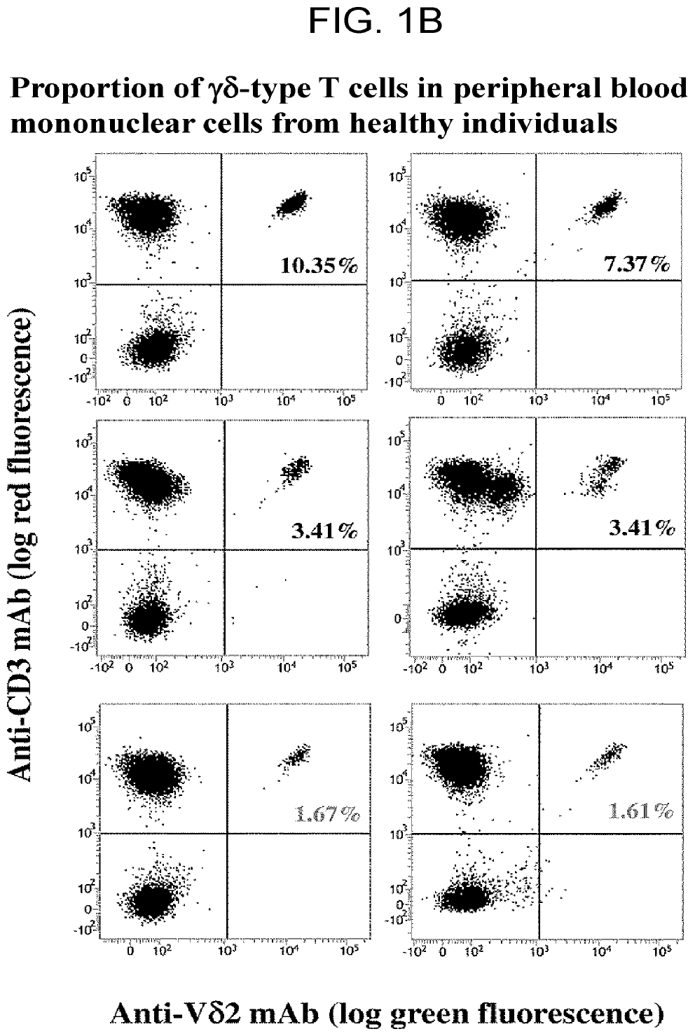

Flow cytometry analysis on proportions of γδ T cells in 12 healthy individuals found that, as previously reported, the proportion of γδ T cells in peripheral blood mononuclear cells was about 3% to 4% on average, and over 10% in some donors. On the other hand, there were as few as two cases of donors with less than 2%, and no donors with less than 1% (FIG. 1).

### (2) Reactivity of γδ T Cells in Healthy Individuals

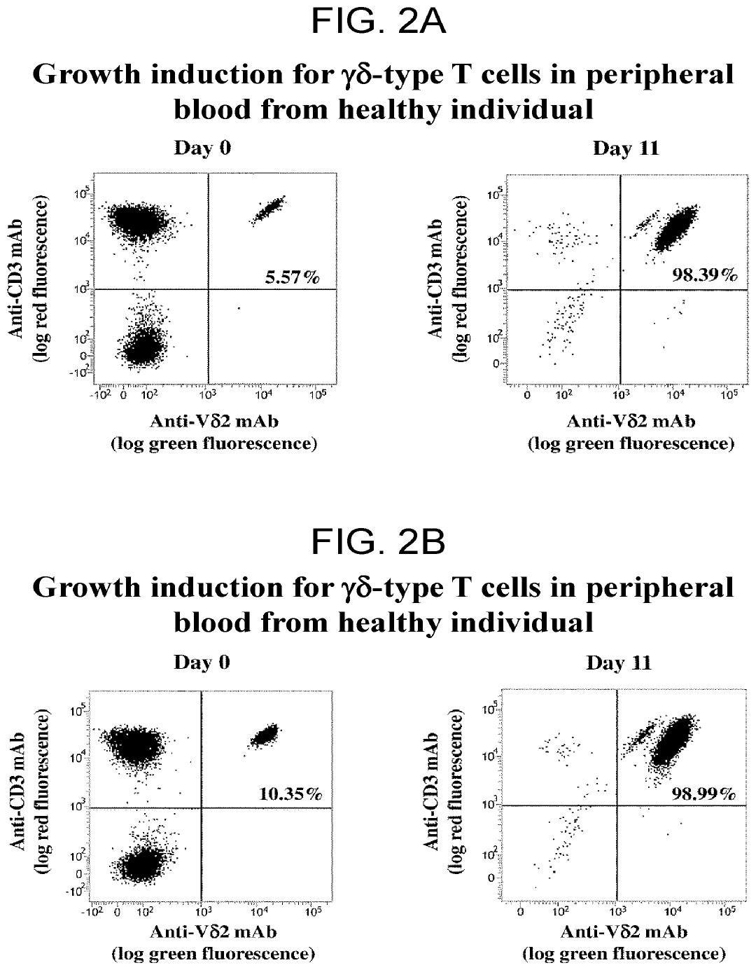

Next, examination was made on reactivity of γδ T cells in healthy individuals. When peripheral blood mononuclear cells of a healthy individual who was found to have a proportion of Vδ2γδ T cells being 5.57% were subject to the action of PTA and cultured together with IL-2 for 11 days, the proportion of γδ T cells increased to 98.39%. Then, the cell count increased by 1000 times or more (FIG. 2(A)).

Similarly, peripheral blood mononuclear cells of a healthy individual who was found to have a proportion of Vδ2γδ T cells being 10.35% were subject to the action of PTA and cultured together with IL-2 for 11 days, the proportion of γδ T cells increased to 98.99%, and the cell count increased by 1000 times or more. Thus, with PTA/IL-2 stimulation, γδ T cells in healthy individuals exhibit a purity of almost 99% and a growth ability of 1000 times or more on Day 11 of culture (FIG. 2(B)).

### (3) Proportion of γδ T Cells in Lung Cancer Patients

Table 1 shows proportions of γ6-type T cells in lung cancer patients. It can be understood that the cases are clearly divided into those with few γδ T cells (LC02, LC05, LC09, LC10) and those with many γδ T cells (LC03, LC04, LC07, LC08).

TABLE 1
γδ-type T cells in lung cancer patients

| | Vδ2/CD3 | Vδ2/Lymphocytes | Vδ2/Lymphocytes/ |
|---|---|---|---|
| Patient | (%) | (%) | 1 × 10e7 PBMC |
| LC01 | 1.89 | 1.43 | 1.43 × 10e5 |
| LC02 | 1.46 | 0.79 | 0.79 × 10e5 |
| LC03 | 14.63 | 10.40 | 10.40 × 10e5 |
| LC04 | 3.76 | 2.91 | 2.91 × 10e5 |
| LC05 | 1.00 | 0.73 | 0.73 × 10e5 |
| LC06 | 1.55 | 1.01 | 1.01 × 10e5 |
| LC07 | 10.43 | 4.14 | 4.14 × 10e5 |
| LC08 | 2.98 | 1.95 | 1.95 × 10e5 |
| LC09 | 0.99 | 0.78 | 0.78 × 10e5 |
| LC10 | 1.23 | 0.89 | 0.89 × 10e5 |
| LC11 | 0.06 | 0.03 | 0.03 × 10e5 |
| LC12 | 0.82 | 0.53 | 0.53 × 10e5 |

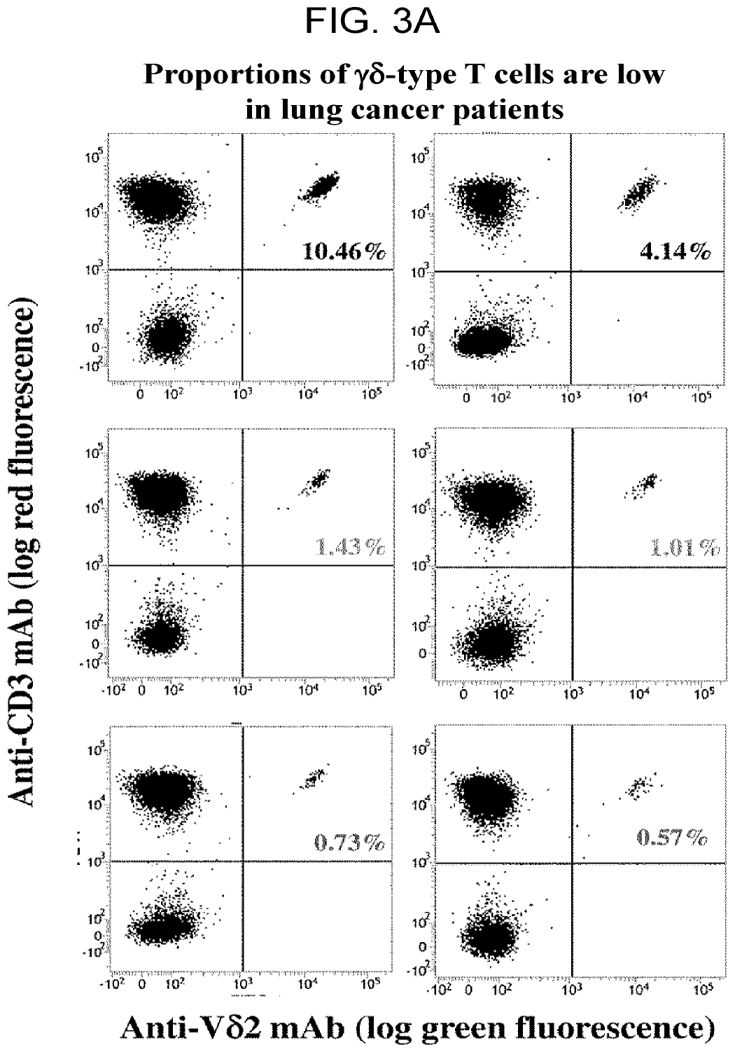

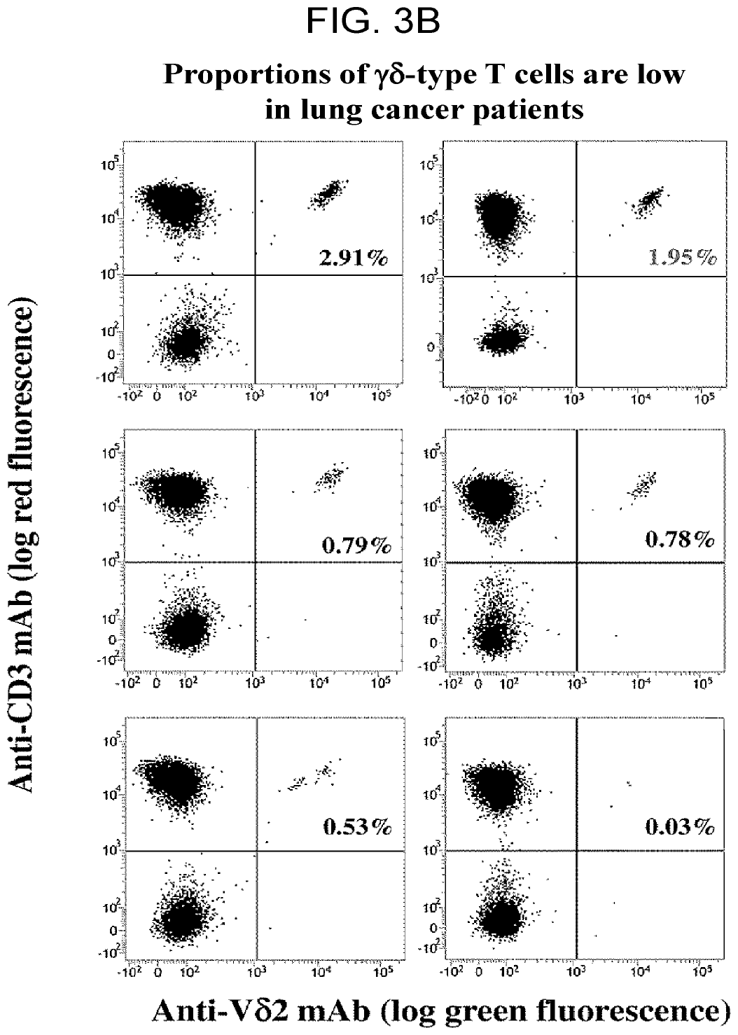

Examination on proportions of γδ T cells in peripheral blood mononuclear cells from the lung cancer patients found significantly decreased proportions of 78 T cells, being 2% or less, in 9 cases of the 12 cases. In details of the cases with a decreased proportion, the proportion was less than 1% in six cases. This indicates the possibility that lung cancer suppressed γδ T cells, causing immunotolerance. Assuming that lung cancer-specific αβ T cells are under immunosuppression similarly to these γδ T cells, it follows that the number of immune effector cells themselves is extremely small, and it is inferred that sufficient immune effector action is not recovered even if immunosuppressive signals are blocked at immune checkpoints (FIG. 3).

### (4) Reactivity of γδ T Cells in Lung Cancer Patients

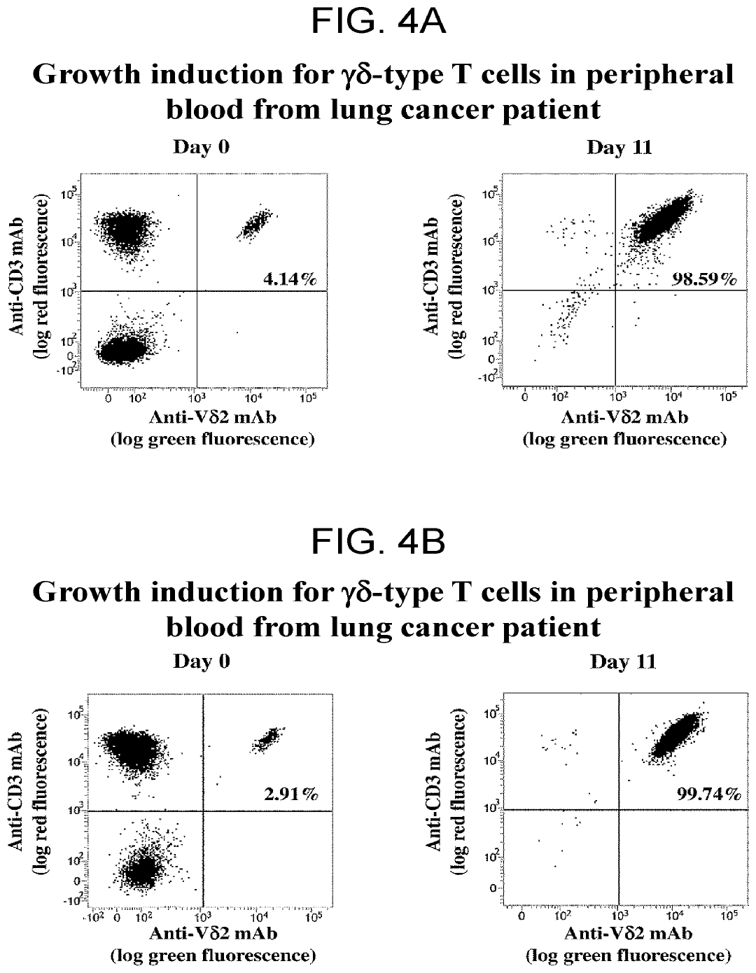

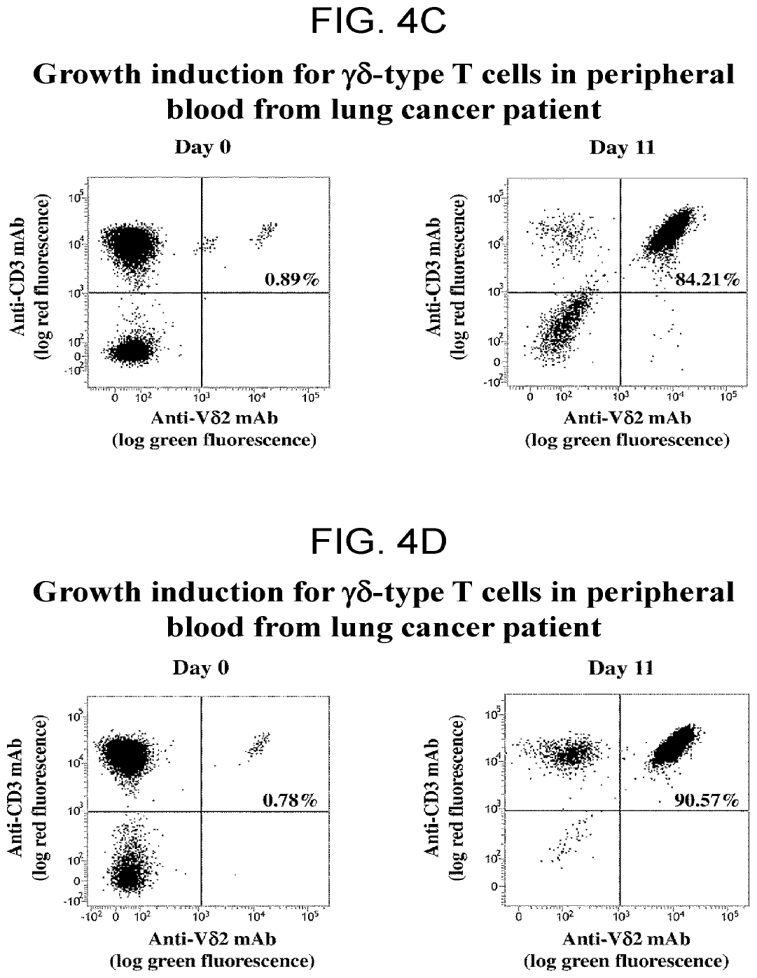

Next, cases of lung cancer patients in which the proportion of γδ T cells was comparable to those in healthy individuals were selected, and examined on growth inducibility for γδ T cells. When peripheral blood mononuclear cells of a lung cancer patient who was found to have a proportion of Vδ2γδ T cells being 4.14% were subject to the action of PTA and cultured together with IL-2 for 11 days, the proportion of γδ T cells increased to 98.59%. Then, the cell count increased by 1000 times or more. Thus, the lung cancer patient with a numerical value close to those of healthy individuals was revealed to have γδ T cell growth inducibility comparable to those of healthy individuals (FIG. 4(A)).

Similarly, examination was made on growth inducibility for peripheral blood γδ T cells in a lung cancer patient who was found to have a proportion of γδ T cells being 2% or more. The result showed that when peripheral blood mononuclear cells of a lung cancer patient who was found to have a proportion of Vδ2γδ T cells being 2.91% were subject to the action of PTA and cultured together with IL-2 for 11 days, the proportion of γδ T cells increased to 99.74%, and the cell count increased by 1000 times or more. It was revealed that also this case exhibited γδ T cell growth inducibility comparable to those of healthy individuals (FIG. 4(B)).

Next, examination was made on growth inducibility for peripheral blood γδ T cells in a lung cancer patient who was found to have a proportion of γδ T cells being less than 1%. When peripheral blood mononuclear cells of a lung cancer patient who was found to have a proportion of Vδ2γδ T cells being 0.89% were subject to the action of PTA and cultured together with IL-2 for 11 days, the proportion of γδ T cells increased only to 84.21%. Thus, the lung cancer patient with a clearly lower proportion of γδ T cells than healthy individuals was revealed to have lower γδ T cell growth inducibility than healthy individuals (FIG. 4(C)).

Similarly, examination was made on growth inducibility for peripheral blood γδ T cells in another lung cancer patient who was found to have a proportion of γδ T cells being less than 1%. The result showed that when peripheral blood mononuclear cells of a lung cancer patient who was found to have a proportion of Vδ2γδ T cells being 0.78% were subject to the action of PTA and cultured together with IL-2 for 11 days, the proportion of γδ T cells increased only to 90.57% (FIG. 4(D)).

As demonstrated, patients who have a low proportion (lower than 1%) of γδ T cells are likely to undergo immunotolerance of γδ T cells. If this is due to some kind of an immunotolerance induction system of tumor cells for immune effector T cells, immunotolerance of αδ T cell specific to tumor antigen peptide is likely to be occurring at the same time. It follows that the state of immunotolerance of αβ T cells specific to tumor antigen peptide can be probably assessed by measuring the proportion of γδ T cells in peripheral blood. Therefore, a criterion for assessing the sensitivity of an immune checkpoint inhibitor may be examination of the proportion of γδ T cells in peripheral blood mononuclear cells, and the proportion of γδ T cells probably serves as a surrogate marker for immune checkpoint inhibitors.

### (5) Relationship Between γδ T Cells and NK Cells

Examination was made on the relationship between 7 T cells and NK cells. First, peripheral blood mononuclear cells of a healthy individual are purified, and stimulated with Zol (Zoledronic acid: Zometa)/IL-2 or Zol/IL-2/IL-18, where Zol is one of nitrogen-containing bisphosphonates (N-BP), according to a previous report (Sigie T. et al., Cancer Immunol Immunother. 2013 April; 62(4):677-87. Epub 2012 Nov. 15.) to examine growth induction for γδ T cells.

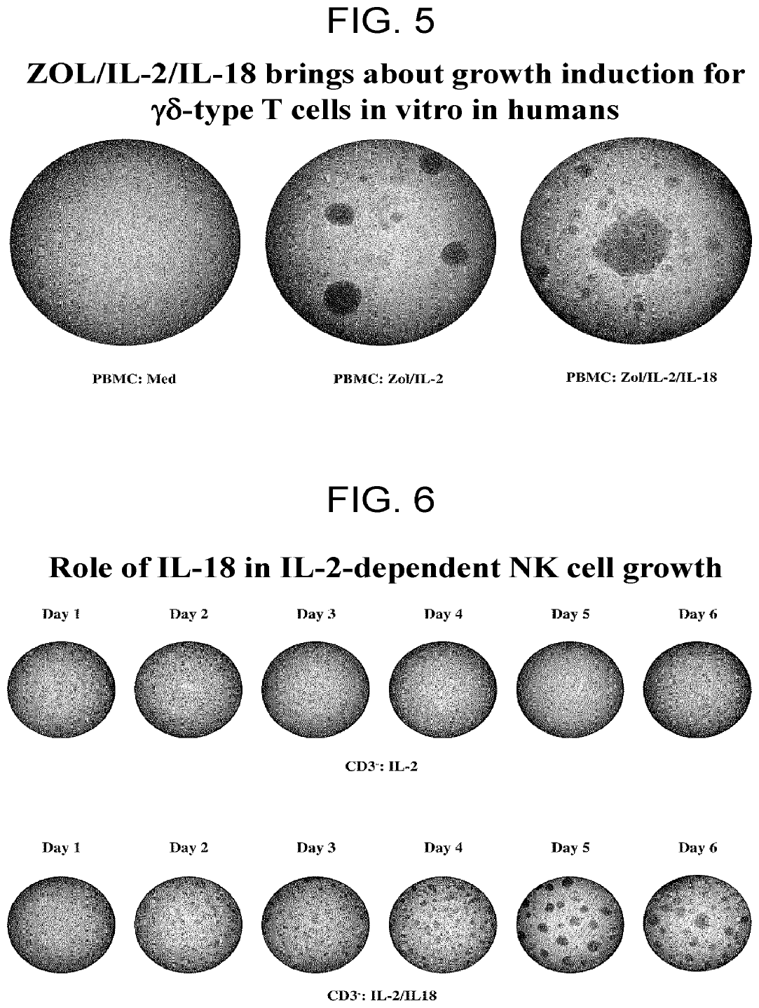

The result found that stimulation with addition of IL-18, which exhibits cell protection action, had clear superiority in growth to mixed stimulation with Zol, which is a stimulation factor for γδ T cells, and IL-2, which is a growth factor therefor (FIG. 5). Examination on how this occurs revealed that growth induction for γδ T cells is suppressed if NK cells are removed from this experimental system. That is, it was revealed that NK cells play an important role in growth induction for human γδ T cells (not shown)

Table 2 shows results after growth induction with Zol/IL-2/IL-18. Like the cases before growth induction, it can be understood that the cases are clearly divided into those with few γδ T cells (LC02, LC05, LC09, LC10) and those with many γδ T cells (LC03, LC04, LC07, LC08)

TABLE 2
γδ-type T cells in lung cancer patients after
growth induction (Zol/IL-2/IL-18 stimulation)

| | Vδ2/CD3 | Vδ2/Lymphocytes | Vδ2/Lymphocytes/ |
|---|---|---|---|
| Patient | (%) | (%) | 1 × 10e7 PBMC |
| LC01 | 97.30 | 93.99 | 3.00 × 10e8 |
| LC02 | 99.26 | 93.88 | 1.30 × 10e8 |
| LC03 | 99.39 | 97.43 | 4.00 × 10e8 |
| LC04 | 99.15 | 97.14 | 14.52 × 10e8 |
| LC05 | 97.82 | 93.71 | 1.05 × 10e8 |
| LC06 | 90.09 | 59.18 | 1.90 × 10e8 |
| LC07 | 99.02 | 95.53 | 4.88 × 10e8 |
| LC08 | 98.45 | 93.18 | 6.50 × 10e8 |
| LC09 | 90.57 | 88.14 | 1.92 × 10e8 |
| LC10 | 95.39 | 60.00 | 1.84 × 10e8 |

### (6) Relationship Between NK Cells and IL-18

Next, examination was made on the relationship between NK cells and IL-18. While NK cells undergo growth induction by IL-2 stimulation, what happens if IL-18 is added thereto was examined. First, CD3-positive cells were removed from human peripheral blood mononuclear cells, and the CD3⁻ cell fraction was stimulated with IL-2 or IL-2/IL-18.

The result found that the IL-2/IL-18 stimulation group exhibited clear superiority in growth induction for NK cells (FIG. 6). Thus, it was revealed that NK cells undergo strong growth induction through IL-2/IL-18 stimulation. Results before and after IL-2/IL-18 stimulation are shown in Table 3 and Table 4, respectively.

TABLE 3
NK cells in healthy individuals before growth induction

| Healthy adult | NK/Lymphocyte | NK/Lymphocyte/1 × 10⁷ PBMC |
|---|---|---|
| HD03 | 10.46 | 10.46 × 10e5 |
| HD04 | 18.18 | 18.18 × 10e5 |
| HD05 | 18.12 | 18.12 × 10e5 |
| HD06 | 13.18 | 13.18 × 10e5 |
| HD07 | 28.42 | 28.42 × 10e5 |
| HD08 | 14.06 | 14.06 × 10e5 |
| HD09 | 4.42 | 4.42 × 10e5 |
| HD10 | 23.58 | 23.58 × 10e5 |
| HD11 | 18.61 | 18.61 × 10e5 |

TABLE 4
NK cells in healthy individuals after growth
induction (IL-2/IL-18 stimulation)

| Healthy adult | NK/Lymphocyte | NK/Lymphocyte/1 × 10e7 PBMC |
|---|---|---|
| HD06 | 96.57 | 1.56 × 10e8 |
| HD07 | 99.07 | 5.77 × 10e8 |
| HD08 | 98.43 | 5.44 × 10e8 |
| HD09 | 96.22 | 2.00 × 10e8 |
| HD10 | 95.37 | 0.83 × 10e8 |
| HD11 | 85.02 | 0.63 × 10e8 |

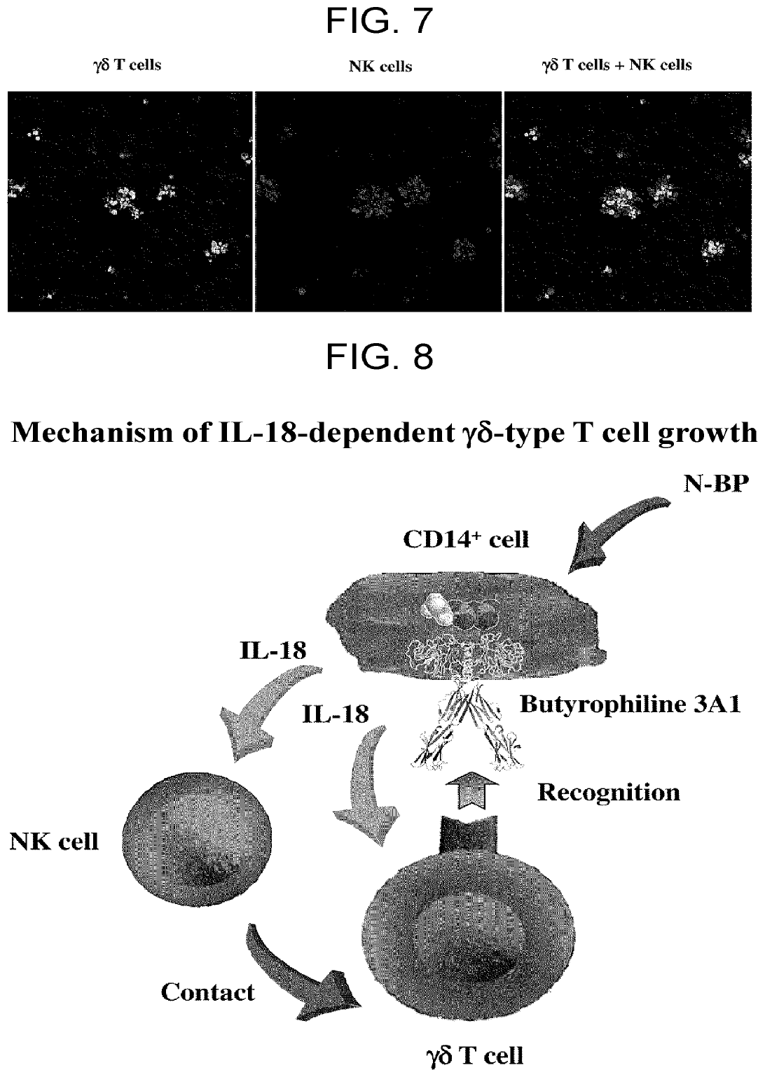

Next, examination was made on how the NK cell growth inducibility of IL-2/IL-18 is associated with growth induction for γδ T cells. First, according to a previous report (see Li et al., PLoS One. 2013 Dec. 20; 8(12), FIG. 2), γδ T cells were labeled with green dye, NK cells that had undergone growth induction through IL-2/IL-18 stimulation were labeled with red dye, and the cells were subjected to mixed culture. The result revealed that γδ T cells and NK cells interacted with each other to form cell clusters (FIG. 7).

3. Discussion

From the above results, the mechanism of growth induction for γδ T cells by N—BP such as Zometa and PTA was expected to be based on the interaction between γδ T cells and NK cells (FIG. 8). Specifically, when N-BP is incorporated in macrophages, which are CD14-positive, the extracellular domain of butyrophilin 3A1 (BTN3A1) changes, and γδ T cells recognize this change in a γδ T cell receptor-dependent manner. After that, a signal is induced from a γδ T cell receptor into γδ T cells and a transcription factor is mobilized to the promoter region for IL-2, and as a result a certain amount of IL-2 production is found. Meanwhile, in macrophages that received N-BP stress, caspase I is activated in an inflammasome-dependent manner to hydrolyze an IL-18 precursor, and causes production of mature IL-18 and extracellular release thereof. Here, NK cells undergo growth induction with IL-2/IL-18. If IL-18 acts also on γδ T cells, on the other hand, expression of LFA-1 and ICAM-1 is induced. A strong interaction between NK cells and γδ T cells occurs via these adhesion molecules, and explosive grow induction for γδ T cells occurs. From these discussions, it is expected to be possible to predict the sensitivity of an immune checkpoint inhibitor by examination primarily on the number (proportion) of γδ T cells and growth induction ability therefor and secondarily on the number (proportion) of NK cells and growth induction ability therefor.

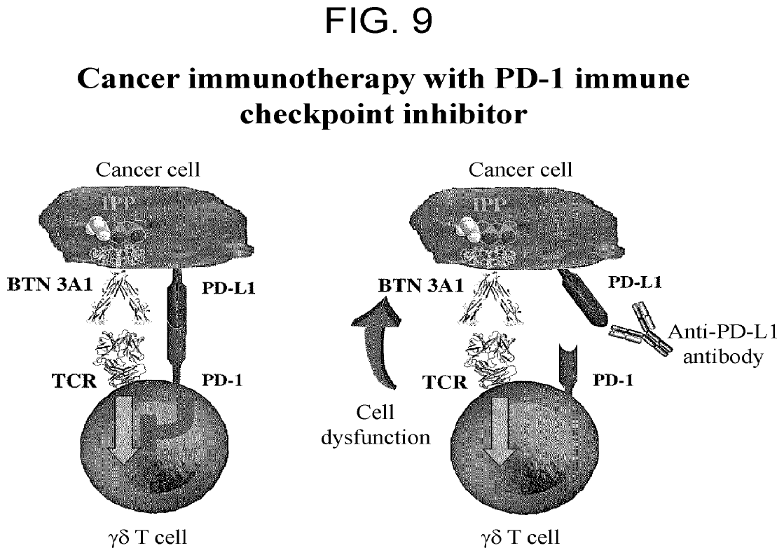

As illustrated in FIG. 9, when a cancer cell expresses a PD-L1 molecule, a negative costimulatory signal is induced to an activated γδ T cell, which is expressing a PD-1 molecule, by the interaction between PD-1 and PD-L1, and antitumor cytotoxicity is suppressed. If an anti-PD-L1 antibody is allowed to act here, the interaction between PD-1 and PD-L1 is blocked and the negative costimulatory signal is cancelled, and hence the γδ T cell becomes able to efficiently disorder cancer cells. If the γδ T cell is under exhaustion, however, the function of the γδ T cell itself has been irreversibly suppressed, and hence the antitumor cytotoxicity of the γδ T cell does not recover even if the interaction between PD-1 and PD-L1 is blocked with a PD-1 immune checkpoint inhibitor. The results of this Example demonstrate that evaluation of the number (proportion) and function of γδ T cells in peripheral blood enables assessment of the state of immunotolerance and prediction of the effect of an immune checkpoint inhibitor.

### Example 2: Objective Response of Nivolumab (Anti-PD-1 Antibody)

The results of Example 1 confirmed that the function and growth ability of immune effector T cells and the growth ability of NK cells are likely to be keys in predicting the antitumor effect of a PD-1 immune checkpoint inhibitor. It follows that if the same immunotolerance induction system works for αβ T cells and γδ T cells, which are included in examples of immune effector T cells, clarifying the state of γδ T cells leads to successful prediction of the state of immunotolerance of αβ T cells.

In view of this, examination was made in this Example on the correlational relationship between the proportion of γδ T cells in peripheral blood mononuclear cells, growth induction ability of antigenic stimulation of γδ T cells, and expression level of PD-1 after growth induction and the objective response and adverse events.

1. Study Design

[Number of facilities] Multicenter study (NAGASAKI University Hospital, Nagasaki Genbaku Hospital)
[Subjects] Lung cancer patients
[Selection criteria]

Employed were patients who had been histologically confirmed to have lung cancer through computed tomography (any T, any N, and M1, stage IV), and

who were 20 to 75 years old with performance status (PS) of 0 and retaining the functions of main tissues, met the study criteria of our institution, and voluntarily submitted a consent form to participate in the study after receiving explanation on the characteristics of the study.

[Exclusion Criteria]

Patients who met any one of the followings were excluded from subjects.

1) Patients having a past history of hypersensitivity to the component of the tested agent
2) Patients who were pregnant or possibly pregnant, or patients who were lactating
3) Other patients who were assessed to be inappropriate as a study subject by a person in charge of the study

[Primary Endpoint]

The first primary endpoint is the correlational relationship between the objective response rate (ORR) and proportions of Vδ2 T cells in a peripheral blood lymphocyte gate and CD3-positive cells in a lung cancer patient to whom nivolumab was administered.

The second primary endpoint is the correlational relationship between the objective response rate (ORR) and the proportion of PD-1-expressing Vδ2 T cells after antigenic stimulation in a lung cancer patient.

[Secondary Endpoint]

The first secondary endpoint is the correlational relationship between the objective response rate (ORR) and the proportion of NK cells in peripheral blood mononuclear cells in a lung cancer patient to whom nivolumab was administered.

The second secondary endpoint is the correlational relationship between the objective response rate (ORR) and the growth rate of NK cells after IL-2/IL-18 stimulation in a lung cancer patient.

[Collection of Specimens]

Blood collection with heparin is performed for 10 ml of peripheral blood for cases with consent to be given administration of the anti-PD-1 antibody nivolumab before administration of nivolumab and 3 months after administration of nivolumab. This blood collection is performed concomitantly with regular blood collection during hospitalization, and no additional puncture is performed for the blood collection.

[Study Outcome Measures]

### (A) Measurement of Tumor Volume and Objective Response Rate (RECIST)

Tumor volume is measured by using MRI. Measurement is performed according to guidelines, and objective response rates (ORR) are individually evaluated according to the RECIST guidelines with use of ultrasonography and clinical evaluation.

(B) Examination of Numbers and Proportions of Vγ2Vδ2 T Cells and CD56-Positive NK Cells in Peripheral Blood Mononuclear Cells Before and after Administration of Anti-PD-1 Antibody Nivolumab

Peripheral blood mononuclear cells (PBMCs) are purified through Ficoll gradient centrifugation (see Example 1), and suspended in 7 ml of YM-AB medium. Of PBMCs suspended in YM-AB medium, 1 ml is subjected to flow cytometry analysis. Specifically, 0.1-ml portions of the cell suspension are seeded in nine wells of a 96-well round-bottom plate, and centrifuged at 1700 rpm and 4° C. for 2 minutes. The supernatant is removed, and the cell pellets are stirred with a Vortex. Thereto, 46 μl of 2% FCS/PBS and any of the followings are added.

### (i) 2% FCS/PBS (4 μl)

(ii) PE-labeled anti-CD3 antibody (2 μl)+2% FCS/PBS (2 μl)
(iii) 2% FCS/PBS (2 μl)+FITC-labeled anti-Vδ2 antibody (2 μl)
(iv) PE-labeled anti-CD3 antibody (2 μl)+FITC-labeled anti-CD4 antibody (2 μl)
(v) PE-labeled anti-CD3 antibody (2 μl)+FITC-labeled anti-CD8 antibody (2 μl)
(vi) PE-labeled anti-CD3 antibody (2 μl)+FITC-labeled anti-Vδ1 antibody (2 μl)
(vii) PE-labeled anti-CD3 antibody (2 μl)+FITC-labeled anti-Vδ2 antibody (2 μl)
(viii) PE-labeled anti-CD25 antibody (2 μl)+FITC-labeled anti-CD4 antibody (2 μl)
(ix) PE-labeled anti-CD56 antibody (2 μl)+FITC-labeled anti-CD3 antibody (2 μl)

After addition of antibodies, the plate is incubated on ice for 15 minutes, and 100 μl of 2% FCS/PBS is added. Thereafter, the plate is centrifuged at 1700 rpm and 4° C. for 2 minutes, and the supernatant is removed. This operation is performed three times in total, and finally 200 μl of 2% FCS/PBS is added, and the resultant is passed through a 70-μm filter membrane and subjected to flow cytometry analysis. Based on this analysis result, the proportion and number of Vγ2Vδ2 T cells and cell surface markers are examined.

(C) Examination on Growth Induction Ability of PTA Stimulation of Peripheral Blood Vδ2 T Cells Before and after Administration of Anti-PD-1 Antibody Nivolumab

For 3 ml of the PBMCs suspended in YM-AB medium, γδ T cell growth test is performed. To 3 ml of the PBMC suspension, 1 mM PTA is added, and the resultant is seeded in two wells of a 24-well plate (1.5 ml/well, two wells), and incubated at 37° C. and 5% CO₂ (Day 0).

IL-2 (final concentration: 100 U/ml) is added to one well, and IL-2 (final concentration: 100 U/ml)+IL-18 (final concentration: 100 ng/ml) is added to the other well (Day 1). IL-2 or IL-2+IL-18 is further added (Day 2-Day 9). On Day 10, cell counts are measured to examine growth induction ability for Vγ2Vδ2 T cells.

(D) Examination on Growth Induction Ability Induced by Peripheral Blood IL-2/IL-18 for NK Cells Before and after Administration of Anti-PD-1 Antibody Nivolumab

For residual 3 ml of the PBMCs suspended in YM-AB medium, NK cell growth test is performed. A 15-ml conical tube containing the cell suspension is centrifuged at 1700 rpm and 4° C. for 5 minutes. Subsequently, the supernatant is removed by suction, and the cell pellets are dispersed and resuspended in 80 μl of PBS/0.5% BSA/2 mM EDTA. Thereto, 20 μl of an anti-CD3 antibody labeled with MACS® Beads (Mylteny Biotec) is added, and the cell suspension is incubated at 4° C. for 15 minutes. Thereto, 2 ml of PBS/0.5% BSA/2 mM EDTA is added, and the cells are lightly suspended. Subsequently, the cell suspension is centrifuged at 300×g and 4° C. for 10 minutes to remove the supernatant. The cell pellets are dispersed, to which 1 ml of PBS/0.5% BSA/2 mM EDTA is added, and the cells are sufficiently suspended. The cell suspension is applied to an LD column (composed of magnetic spheres) equilibrated with PBS/0.5% BSA/2 mM EDTA. CD3-negative cells are eluted twice with 1 ml of PBS/0.5% BSA/2 mM EDTA. Centrifugation is performed at 1700 rpm and 4° C. for 5 minutes, and the supernatant is discarded and the residual supernatant is removed by suction with an aspirator. Subsequently, the cell pellets are dispersed, and the CD3 cells are suspended in 1.5 ml of YM-AB medium. The cells are seeded in a 24-well plate, IL-2 and IL-18 are added to final concentrations of 100 U/ml and 100 ng/ml, respectively, and the resultant is incubated at 37° C. and 5% CO₂ (Day 0) IL-2 and IL-18 are added to the medium every day (Day 2-Day 9). On Day 10, cell counts are measured to examine growth induction ability for NK cells.

(E) Analysis of Surface Markers of Vδ2 T Cells Including PD-1 after Growth Induction (Method: See (F))

Vγ2Vδ2 T cells subjected to growth induction with PTA are collected on Day 10, and cell surface markers are examined by flow cytometry. Specifically, 0.1-ml portions of the cell suspension are seeded in seven wells of a 96-well round-bottom plate, and centrifuged at 1700 rpm and 4° C. for 2 minutes. The supernatant is removed, and the cell pellets are stirred with a Vortex. Thereto, 46 μl of 2% FCS/PBS and any of the followings are added.

### (i) 2% FCS/PBS (4 μl)

(ii) PE-labeled anti-CD3 antibody (2 μl)+2% FCS/PBS (2 μl)
(iii) 2% FCS/PBS (2 μl)+FITC-labeled anti-Vδ2 antibody (2 μl)
(iv) PE-labeled anti-CD3 antibody (2 μl)+FITC-labeled anti-Vδ2 antibody (2 μl)
(v) PE-labeled anti-CD3 antibody (2 μl)+FITC-labeled anti-CD4 antibody (2 μl)
(vi) PE-labeled anti-CD3 antibody (2 μl)+FITC-labeled anti-CD8 antibody (2 μl)
(vii) PE-labeled anti-CD56 antibody (2 μl)+FITC-labeled anti-Vδ2 antibody (2 μl)
(viii) 2% PE-labeled anti-NKG2D antibody (2 μl)+FITC-labeled anti-Vδ2 antibody (2 μl)
(ix) PE-labeled anti-DNAM-1 antibody (2 μl)+FITC-labeled anti-Vδ2 antibody (2 μl)
(x) PE-labeled anti-FasL (2 μl)+FITC-labeled anti-Vδ2 antibody (2 μl)
(xi) PE-labeled anti-TRAIL antibody (2 μl)+FITC-labeled anti-Vδ2 antibody (2 μl)
(xii) PE-labeled anti-CD16 antibody (2 μl)+FITC-labeled anti-Vδ2 antibody (2 μl)
(xiii) non-labeled anti-PD-1 antibody (2 μl)+RPE-labeled anti-mouse IgG antibody (2 μl)+FITC-labeled anti-Vδ2 antibody (2 μl)

After addition of antibodies, the plate is incubated on ice for 15 minutes, and 100 μl of 2% FCS/PBS is added. Thereafter, the plate is centrifuged at 1700 rpm and 4° C. for 2 minutes, and the supernatant is removed. This operation is performed three times in total, and finally 200 μl of 2% FCS/PBS is added, and the resultant is passed through a 70-μm filter membrane and subjected to flow cytometry analysis. Based on this analysis result, the proportion and number of Vγ2Vδ2 T cells and cell surface markers are examined.

(F) Analysis of Surface Markers of NK Cells after Growth Induction

On Day 10, cells are collected and examination is made on cell surface markers by flow cytometry. Specifically, 0.1-ml portions of the cell suspension are seeded in seven wells of a 96-well round-bottom plate, and centrifuged at 1700 rpm and 4° C. for 2 minutes. The supernatant is removed, and the cell pellets are stirred with a Vortex. Thereto, 46 μl of 2% FCS/PBS and any of the followings are added.

### (i) 2% FCS/PBS (4 μl)

(ii) PE-labeled anti-CD56 antibody (2 μl)+FITC-labeled anti-CD3 antibody (2 μl)
(iii) PE-labeled anti-NKG2D antibody (2 μl)+FITC-labeled anti-CD56 antibody (2 μl)
(iv) PE-labeled anti-DNAM-1 antibody (2 μl)+FITC-labeled anti-CD56 antibody (2 μl)
(v) PE-labeled anti-FasL (2 μl)+FITC-labeled anti-CD56 antibody (2 μl)
(vi) PE-labeled anti-TRAIL antibody (2 μl)+FITC-labeled anti-CD56 antibody (2 μl)
(vii) PE-labeled anti-CD16 antibody (2 μl)+FITC-labeled anti-CD56 antibody (2 μl)

After addition of antibodies, the plate is incubated on ice for 15 minutes, and 100 μl of 2% FCS/PBS is added. Thereafter, the plate is centrifuged at 1700 rpm and 4° C. for 2 minutes, and the supernatant is removed. This operation is performed three times in total, and finally 200 μl of 2% FCS/PBS is added, and the resultant is passed through a 70-km filter membrane and subjected to flow cytometry analysis on cell surface markers of Vγ2Vδ2 T cells. Based on this analysis result, the proportion and number of NK cells and cell surface markers are examined.

(G) Examination on Usefulness of PD-1 Immune Checkpoint Inhibitor to Vδ2 T Cells after Growth Induction

Cytotoxicity assay is performed for Vγ2Vδ2 T cells subjected to growth induction. Daudi/hPD-L1, a cell line derived from human Daudi Burkitt's lymphoma with forced expression of human PD-L1, is used as target cells, and the mouse anti-human PD-L1 antibody 27A2 is used as a PD-1 immune checkpoint inhibitor.

First, Daudi/hPD-L1 is suspended in 30 ml of RPMI1640 medium, and cultured in a 75-cm² flask at 37° C. and 5% CO₂. The cell count is measured, and 1×10⁶ cells are transferred into four 15-ml conical tubes. The cell suspensions are centrifuged at 1700 rpm and 4° C. for 5 minutes, each supernatant is removed by suction, and the cell pellets are dispersed. In each of tubes 1 and 2, cells are suspended in 1 ml of RPMI1640 medium to prepare a cell suspension at 1×10⁶ cells/ml. To each of tubes 3 and 4, 1 ml of 100 nM PTA solution is added and the resultant is sufficiently suspended. Incubation is performed in an atmosphere at 37° C. and 5% CO₂ for 1 hour 45 minutes. To tube 4, 2 μl of 1 mg/ml mouse anti-human PD-L1 monoclonal antibody 27A2 is added to a final concentration of 0.5 μg/ml. Further, the tubes are incubated in an atmosphere at 37° C. and 5% CO₂ for 15 minutes. Next, 2.5 μl of DMSO is added to tube 1, and 2.5 μl of the terpyridine derivative Ch46 (bis(butylyloxymethyl) 4′-(hydroxymethyl)-2,2′:6′,2″-terpyridine-6,6′-dicarboxylate: see WO 2015/152111, Example 8) is added to each of tubes 2 to 4, and the tubes are incubated in an atmosphere at 37° C. and 5% CO₂ for 15 minutes. Next, the tubes are centrifuged at 1700 rpm and 4° C. for 5 minutes to remove each supernatant. The cell pellets are tapped, to which 2 ml of RPMI1640 medium is added and cells are sufficiently suspended, and then this operation is repeated three times to wash the cells. The cells are suspended with 5 ml of RPMI1640 medium, and 2 ml of this cell suspension is transferred into a new 15-ml conical tube, and 6 ml of RPMI1640 medium is added thereto to a final cell concentration of 5×10⁴ cells/ml=5×10³ cells/100 μl. Into a 15-ml conical tube, 1×10⁷ Vγ2Vδ2 T cells in RPMI1640 medium are transferred. Centrifugation is performed at 1700 rpm and 4° C. for 5 minutes, the supernatant is removed by suction, the cells are added to 5 ml of RPMI1640 medium and sufficiently suspended, and then serial dilution is performed as follows to prepare cell suspensions.

5 ml (Vγ2Vδ2T cells):2×10⁶/ml:40:1(E/T ratio)

2 ml (2×10⁶/ml)+2 ml (RPMI): 1×10⁶/ml:20:1 (E/T ratio)

2 ml (1×10⁶/ml)+2 ml (RPMI):5×10⁵/ml:10:1 (E/T ratio)

2 ml (5×10⁵/ml)+2 ml (RPMI):2.5×10⁵/ml:5:1 (E/T ratio)

2 ml (2.5×10⁵/ml)+2 ml (RPMI):1.25×10⁵/ml:2.5:1(E/T ratio)

2 ml (1.25×10⁵/ml)+2 ml (RPMI):6.25×10⁴/ml:1.25:1(E/T ratio)

2 ml (6.25×10⁵/ml)+2 ml (RPMI):3.125×10⁴/ml:0.625:1(E/T ratio)

2 ml (RPMI):0/ml:0:1(E/T ratio)

Next, each of 100 μl of Vγ2Vδ2 T cell suspension (for study) and 100 μl of RPMI1640 medium (for measurement of natural leakage) or 90 μl of RPMI1640 medium (for measurement of maximum leakage) is added to three wells of a 96-well round-bottom plate. Thereto, 100 μl of Daudi/hPD-L1 cells is added, and centrifugation is performed at 500 rpm and room temperature for 2 minutes and incubation is performed in an atmosphere at 37° C. and 5% CO₂ for 15 minutes, and 10 μl of 0.125% digitonin (in 19% DMSO (MiliQ solution)) is then added to each of the wells for measurement of maximum leakage, and pipetting is sufficiently performed. The plate is further incubated in an atmosphere at 37° C. and 5% CO₂ for 20 minutes or longer, and centrifuged at 1700 rpm and 4° C. for 2 minutes. Next, 25 μl of each supernatant is transferred into a new 96-well round-bottom plate, and sufficiently mixed with 250 μl of Eu solution. To a new plate for fluorescence measurement, 200 μl of this is transferred, and time-resolved fluorescence is measured. Based on these results, examination is made on the influence of the PD-1 immune checkpoint inhibitor on the antitumor effect of Vγ2Vδ2 T cells subjected to growth culture against the PD-L1-expressing tumor cell line.

(H) Study of Correlation Between NK Cells after Growth Induction and Usefulness of PD-1 Immune Checkpoint Inhibitor

Cytotoxicity assay is performed for NK cells subjected to growth induction. Time-resolved fluorescence is measured with the same procedure as in (G) above except that the human myeloma-derived cell line K562 is used as target cells and that NK cells are used in place of Vγ2Vδ2 T cells, and based on the result, examination is made on the antitumor effect of NK cells subjected to growth culture against the K562 cell line.

[Statistical Analysis]

Before and after administration of nivolumab, summary statistics (e.g., number of cases, mean, standard deviation, minimum, interquartile range, median, maximum) are determined for the biomarkers shown in the above outcome measures. Cases are divided into two groups by the presence or absence of the objective response of nivolumab, and summary statistics are determined for each biomarker. Based on these results, examination is made on the correlational relationship between the proportion of γδ T cells in peripheral blood mononuclear cells, the growth induction ability of antigenic stimulation of γδ T cells, and the expression level of PD-1 after growth induction, and the objective response. Further, examination is made on the correlational relationship between the proportion of NK cells and growth inducibility therefor, and the objective response. Next, an ROC curve is prepared with each biomarker to estimate a cutoff value for the objective response of nivolumab. If multiple factors are suspected to be involved in the objective response of nivolumab, a multifactor ROC curve is determined with each biomarker by using a Logistic model, and a cutoff value with multiple factors is estimated.

[Results]

The results are shown in the following table.

TABLE 5
Staining before treatment, day 0

| | Day 0 (PBMC) | | | | | | | | | | |
|---|---|---|---|---|---|---|---|---|---|---|---|
| | CD3+ | CD3+ | | CD3+ | CD3+ | | CD3+ | CD3+ | | CD3+ | CD3+ |
| | CD4+/ | CD4−/ | CD4/ | CD8+/ | CD8−/ | CD8/ | Vd1+/ | Vd1−/ | Vd1/ | Vd2+/ | Vd2−/ |
| | All | All | CD3 | All | All | CD3 | All | All | CD3 | All | All |
| Patient | (%) | (%) | (%) | (%) | (%) | (%) | (%) | (%) | (%) | (%) | (%) |
| TR01 | 43.9 | 36.5 | 54.60 | 20.0 | 57.4 | 25.84 | 0.31 | 79.9 | 0.39 | 4.86 | 75.1 |
| TR02 | 37.2 | 24.2 | 60.59 | 19.5 | 46.2 | 29.68 | 0.11 | 71.8 | 0.15 | 2.74 | 70.6 |
| TR03 | 13.6 | 8.4 | 61.87 | 8.16 | 14.0 | 36.82 | 0.23 | 21.2 | 1.07 | 0.12 | 21.4 |
| TR04 | 48.2 | 9.4 | 83.65 | 8.59 | 51.6 | 14.27 | 0.66 | 58.9 | 1.11 | 0.74 | 60.9 |
| TR05 | 29.8 | 28.6 | 51.03 | 10.1 | 40.4 | 20.00 | 1.14 | 58.9 | 1.90 | 0.44 | 59.2 |
| TR06 | 16.6 | 10.1 | 62.17 | 2.33 | 22.9 | 9.24 | 2.33 | 27.3 | 7.86 | 0.023 | 29.8 |
| TR07 | 30.7 | 20.9 | 59.50 | 9.10 | 39.4 | 18.76 | 1.17 | 52.0 | 2.20 | 0.51 | 53.5 |
| TR08 | 36.6 | 12.7 | 74.24 | 10.2 | 36.9 | 21.66 | 0.017 | 44.9 | 0.04 | 1.04 | 44.1 |
| TR09 | 40.6 | 12.7 | 76.17 | 11.7 | 42.1 | 21.75 | 0.40 | 52.8 | 0.75 | 0.52 | 53.6 |
| TR10 | 27.4 | 34.8 | 44.05 | 31.4 | 28.5 | 52.42 | 1.06 | 61.2 | 1.70 | 0.60 | 61.0 |
| TR11 | 41.4 | 18.5 | 69.12 | 17.4 | 42.6 | 29.00 | 0.24 | 59.9 | 0.40 | 1.67 | 58.8 |
| TR12 | 23.6 | 18.7 | 55.79 | 16.1 | 27.1 | 37.27 | 1.88 | 41.3 | 4.35 | 0.92 | 41.1 |
| TR13 | 48.5 | 23.7 | 67.17 | 23.0 | 48.7 | 32.08 | 0.81 | 71.2 | 1.12 | 0.34 | 72.2 |
| Average | 33.70 | | 63.07 | 14.43 | | 26.83 | 0.80 | | 1.77 | 1.12 | |

| | Day 0 (PBMC) | | | | | Day 0 (CD3+ cells) | | | |
|---|---|---|---|---|---|---|---|---|---|
| | | CD25+ | CD25− | | CD56+ | CD56+ | CD56+ | CD56+ | |
| | Vd2/ | CD4+/ | CD4+/ | CD25/ | CD3−/ | CD3−/ | CD16+/ | CD16−/ | CD16/ |
| | CD3 | All | All | CD4 | All | All | All | All | CD56 |
| Patient | (%) | (%) | (%) | (%) | (%) | (%) | (%) | (%) | (%) |
| TR01 | 6.08 | 10.4 | 33.7 | 23.58 | 9.39 | 46.9 | 44.5 | 2.24 | 95.21 |
| TR02 | 3.74 | 6.54 | 37.3 | 14.92 | 6.80 | 37.3 | 32.7 | 5.19 | 86.30 |
| TR03 | 0.56 | 4.19 | 9.5 | 30.56 | 7.56 | 9.48 | 8.94 | 0.27 | 97.07 |
| TR04 | 1.20 | 27.1 | 23.5 | 53.56 | 5.30 | 16.1 | 9.86 | 2.80 | 77.88 |
| TR05 | 0.74 | 4.43 | 26.4 | 14.37 | 12.6 | 31.8 | 30.2 | 1.58 | 95.03 |
| TR06 | 0.08 | 1.45 | 16.2 | 8.22 | 40.7 | 61.5 | 60.2 | 0.42 | 99.31 |
| TR07 | 0.94 | 8.10 | 24.1 | 25.16 | 15.6 | 32.7 | 29.0 | 4.28 | 87.14 |
| TR08 | 2.30 | 4.35 | 26.8 | 13.96 | 26.2 | 43.5 | 34.3 | 1.07 | 96.97 |
| TR09 | 0.96 | 7.42 | 31.2 | 19.21 | 27.6 | 38.1 | 38.4 | 1.38 | 96.53 |
| TR10 | 0.97 | 4.99 | 20.6 | 19.50 | 12.1 | 33.6 | 32.9 | 1.38 | 95.97 |
| TR11 | 2.76 | 13.1 | 28.3 | 31.64 | 19.80 | 53.1 | 49.5 | 2.73 | 94.77 |
| TR12 | 2.19 | 7.61 | 17.2 | 30.67 | 38.7 | 67.2 | 65.5 | 0.90 | 98.64 |
| TR13 | 0.47 | 9.20 | 40.1 | 18.66 | 15.3 | 55.7 | 53.6 | 2.13 | 96.18 |
| Average | 1.77 | 8.38 | | 23.39 | 18.28 | 40.54 | 37.66 | | 93.62 |

The onset of interstitial pneumonia was found for TR01, TR02, TR03, and TR07 among 13 subjects. Among them, TR01 and TR02 were presenting with DAD (diffuse alveolar damage) and underwent acute exacerbation in contrast to the other two cases (OP (organizing pneumonia)), and one case resulted in death in spite of discontinuation of administration. In contrast to TR03 and TR07, TR01 and TR02 each had a high proportion of γδ T cells to peripheral blood mononuclear cells (CD3⁺Vδ2⁺/All) and a high proportion of Vδ2⁺ cells to T cells (Vδ2⁺/CD3⁺), and statistical analysis confirmed complete separation of data.

From these results, it was expected that interstitial pneumonia involving DAD can be distinguished from other types of interstitial pneumonia and the risk of onset can be predicted by using the cell count or proportion of Vδ2⁺γδ T cells to peripheral blood mononuclear cells as an index. This enables safe treatment with an immune checkpoint inhibitor, including identifying patients having a risk of the occurrence of a serious adverse event before administration and excluding them from therapeutic targets before administration, or performing pretreatment for them. Although evaluation was conducted based on peripheral blood mononuclear cells here, the cell count or proportion of Vδ2⁺ cells to peripheral blood T cells can be used as an index in the case of a sample with many CD3-V6 cells.

### INDUSTRIAL APPLICABILITY

The present invention is useful for achievement of precision medicine because the risk of onset of severe interstitial pneumonia caused by an immune checkpoint inhibitor is predicted before administration and whether the treatment would be appropriate or not is assessed.

All of the publications, patents, and patent applications cited herein are intended to be directly incorporated herein by reference.

1. A method for predicting a risk of onset of severe interstitial pneumonia caused by an immune checkpoint inhibitor, the method comprising:
measuring:
(a) cell count or proportion of Vδ2⁺γδ T cells in peripheral blood mononuclear cells isolated from a subject;
(b) cell count or proportion of Vδ2^{|}γδ T cells after antigenic stimulation in peripheral blood mononuclear cells isolated from a subject;
(c) cell count or proportion of Vδ2⁺γδ T cells in peripheral blood T cells isolated from a subject; and/or
(d) cell count or proportion of Vδ2⁺γδ T cells after antigenic stimulation in peripheral blood T cells isolated from a subject; and
assessing the risk of onset of severe interstitial pneumonia based on the cell count or proportion.

2. The method of claim 1, wherein if the cell count or proportion is equal to or more than a cutoff value, the subject is predicted to have a high risk of onset of severe interstitial pneumonia.

3. The method of claim 1, wherein if cells after antigenic stimulation aggregate, the subject is predicted to have a high risk of onset of severe interstitial pneumonia.

4. A method for assessing whether a treatment with an immune checkpoint inhibitor would be appropriate or not, the method comprising:
carrying out the method of claim 1, to obtain a prediction of onset risk; and
assessing whether treatment with an immune checkpoint inhibitor would be appropriate or not based on the prediction.

5. The method of claim 1, wherein the antigenic stimulation of γδ T cells is carried out by an antigen comprising IL-2, a phosphomonoester, pyrophosphomonoester, triphosphomonoester, tetraphosphomonoester, triphosphodiester, tetraphosphodiester, nitrogen comprising bisphosphonate, alkylamine, alkyl alcohol, alkenyl alcohol, isoprenyl alcohol, human-derived tumor cell, or a mixture of two or more of any of these.

6. The method of claim 5, wherein in addition to the antigenic stimulation, γδ T cells are stimulated by IL-18, IL-2, IL-7, IL-12, IL-15, IL-21, IL-23, interferon-γ, and/or peripheral blood-conditioned medium.

7. The method of claim 1, wherein the cell count or proportion is measured using flow cytometry or image cytometry.

8. A kit for assessing whether treatment with an immune checkpoint inhibitor would be appropriate or not, the kit comprising:
(i) an anti-CD3 antibody; and
(ii) an anti-Vδ2 antibody.

9. The kit of claim 8, further comprising:
(iii) a pyrophosphomonoester derivative or a nitrogen comprising bisphosphonate derivative; and/or
(iv) IL-18.

10. The kit of claim 8, further comprising:
(iii) a pyrophosphomonoester derivative.

11. The kit of claim 8, further comprising:
(iii) a nitrogen-comprising bisphosphonate derivative.

12. The kit of claim 8, further comprising:
(iv) IL-18.

13. The kit of claim 8, further comprising:
(iii) a pyrophosphomonoester derivative or a nitrogen-comprising bisphosphonate derivative; and
(iv) IL-18.
